(19) United States
(12) Patent Application Publication
(10) Pub. No.: US 2024/0226120 A1
(43) Pub. Date: Jul. 11, 2024

(54) INTERACTION OF SARS-COV-2 PROTEINS WITH MOLECULAR AND CELLULAR MECHANISMS OF HOST CELLS AND FORMULATIONS TO TREAT COVID-19

(71) Applicants: Shreema Merchant; Manit Patel, Surrey (CA); AKSEERA PHARMA CORP., Vancouver (CA)

(72) Inventors: Shreema Merchant, Surrey (CA); Manit Patel, Surrey (CA); Robin Elaine Duncan, Waterloo (CA); Vishal Anant Jadhav, Mumbai (IN); Maria Fernanda de Andrade Fernandes, Kitchener (CA)

(21) Appl. No.: 17/915,862

(22) PCT Filed: Mar. 30, 2021
(86) PCT No.: PCT/IN21/50325

§ 371 Date: Sep. 29, 2022

(30) Foreign Application Priority Data
Mar. 29, 2020 (IN) 202021013770
Jul. 18, 2020 (IN) 202021030633
Dec. 12, 2020 (IN) 202021054151

(51) Int. Cl. A61K 31/00; A61P 31/14; A61P 37/04

(52) U.S. Cl. CPC A61K 31/658; A61P 31/14; A61P 37/04

(57) ABSTRACT

The present invention provides pharmaceutical compositions and methods of treating Covid-19 infectious disease. The present invention also provides pharmaceutical compositions and methods of prophylaxis or prophylactic treatment of Covid-19 infectious disease. The said methods involve administering compositions comprising therapeutically effective amount of Cannabidiol thereby causing enhancement/augmentation of innate immunity of the patient/mammal/human.

### FIELD OF THE INVENTION

The present invention provides pharmaceutical compositions and methods of treating Covid-19 infectious disease. The present invention also provides pharmaceutical compositions and methods of prophylaxis or prophylactic treatment of Covid-19 infectious disease.

### SUMMARY OF THE INVENTION

Treatment or prophylaxis of Covid-19 infectious disease is extremely challenging. This is more so because SARS-COV-2 has many variants and some of the variants have
i) increased transmissibility,
ii) increased virulence, and
iii) reduced effectiveness of vaccines.

Under the first aspect, the present invention provides pharmaceutical compositions and methods for treating Covid-19 infectious disease comprising administering to patient such pharmaceutical compositions comprising therapeutically effective amount of Cannabidiol wherein such administration of said pharmaceutical composition to the said patient produces an enhancement/augmentation of innate immunity of the patient due to at least one of the following effects,
i) infected patient cells undergo apoptosis early after infection;
ii) induction of interferon transcription in the patient;
iii) induction of interferon-induced antiviral effectors in the patient.

Under the second aspect, the present invention also provides pharmaceutical compositions and methods for prophylaxis or prophylactic treatment of Covid-19 infectious disease comprising administering to mammal/human such pharmaceutical compositions comprising therapeutically effective amount of Cannabidiol wherein such administration of said pharmaceutical composition to the said mammal/human produces an enhancement/augmentation of innate immunity of the patient due to at least one of the following effects,
i) induction of interferon transcription in the patient;
ii) induction of interferon-induced antiviral effectors in the patient.

Under the third aspect, the invention provides a pharmaceutical composition and method of administering pharmaceutical composition comprising therapeutically effective amount of Cannabidiol for preventing or reducing mutation of Sars-Cov-2 virus in a patient by administration of said pharmaceutical composition to the said patient suffering from Covid-19 by causing infected patient cells to undergo apoptosis early after infection which renders them not available to the virus for mutation.

Under the fourth aspect, the invention provides a pharmaceutical composition and methods for administering pharmaceutical composition comprising therapeutically effective amount of Cannabidiol for use in preventing or better preparing for Covid-19 infectious disease in mammals/humans who are about to get infected wherein administration of said pharmaceutical composition to the mammal/human produces an enhancement/augmentation of innate immunity in such mammal/human due to at least one of the following effects,
i) induction of interferon transcription in the mammal/human;
iii) induction of interferon-induced antiviral effectors in the mammal/human;

wherein such induction is not associated with apoptosis of cells in the beginning enabling cells to get primed/prepared for viral threat and wherein such cells are better able to prepare for infection including increase in the infectious dose of virions than usual dose and wherein the cells undergo apoptosis early after infection which renders the cells not available to the virus for replication and/or mutation.

### BRIEF DESCRIPTION OF FIGURES

FIGS. 1-5 depict cell proliferation rates which were measured by incorporating and quantifying bromodeoxyuridine (BrdU) into DNA of actively proliferating cells. The absorbance values are measured by ELISA assay with a BioTek Synergy H1 Hybrid Multi-Mode Microplate reader assay at 370 nm (reference wavelength: approx. 492 nm).

HEK293 (human embryonic kidney) cells were seeded in 96 well plates, then transfected with plasmids expressing an empty control vector (pCMV-3Tag-3a) or vectors expressing the viral Orf8, Orf10 or M proteins. Untransfected control cells have also been tested, but did not differ significantly from pCMV controls.

A few hours later the cells were treated with 1 μM of the cannabinoid, then grown for 24 hours, and assayed using a colorimetric ELISA that detects BrdU incorporation.

The inventors have run 2-way ANOVA. This has been tested in multiple separate assays on different days/weeks, with n=5 to 6 biological replicates (where separate passages of cells were considered different biological replicates). Each biological replicate was seeded in 2 to 6 technical replicates per plate, and those were averaged at each trial to give n=1 for that biological replicate in that trial.

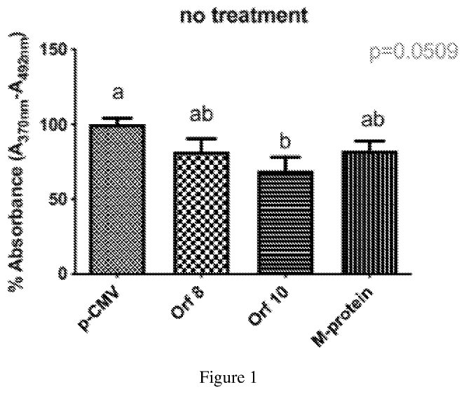

FIG. 1 provides a “no treatment” condition where HEK293 (human embryonic kidney) cells are transfected with plasmids expressing an empty control vector (pCMV-3Tag-3a) or vectors expressing the viral Orf8, Orf10 or M proteins.

Untransfected control cells (not shown in the figure) have also been tested but did not differ significantly from pCMV controls.

The viral plasmids appear to cause only a minor decrease in cell proliferation (or, possibly increases in cell death, or both). This minor decrease which is even less considering error bars is not significant to conclude impact of viral plasmids on cell proliferation. These data were not normalized to account for differences in the number of cells per well, hence it is essential to do normalization before any conclusions can be drawn from these data.

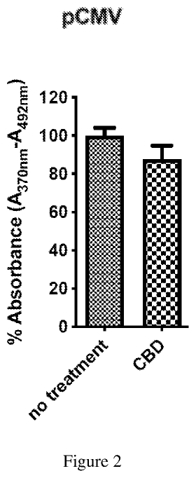

FIG. 2 provides a “control” condition where HEK293 (human embryonic kidney) cells are transfected with plasmids expressing an empty control vector (pCMV-3Tag-3a) and further treated with Cannabidiol.

Cannabinoids did not significantly affect the incorporation of BrdU into cells transfected with the control plasmid.

They also did not affect the growth of untransfected control cells, or cells transfected with another control vector, pEGFP-N1 (data not shown in this figure).

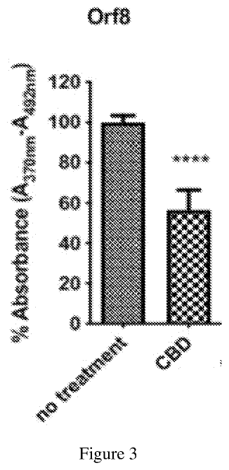

FIG. 3 provides a condition where HEK293 (human embryonic kidney) cells are transfected with plasmids expressing the viral Orf8 protein and are further treated with Cannabidiol.

Surprisingly, significant reduction in mean cell proliferation was observed, although since this data was not normalized to the number of cells that were present in the well, no conclusions can be drawn. This decrease could have been due to a decrease in cell proliferation, or a decrease in cell number, or both.

In cells expressing Orf8, BrdU incorporation was significantly reduced by treatment with any cannabinoid relative to untreated cells. This could reflect a significant decrease in mean cell proliferation, or the same rate of cell proliferation, but a decrease in cell number. Analysis is by 1-way ANOVA with Tukey's multiple comparison's test, where columns with different superscripts are significantly different, ***P<0.001, ****P<0.0001.

In cells expressing Orf8 and treated with CBD, mean BrdU incorporation was 43.52% lower than in cells expressing Orf8 but untreated with cannabinoids.

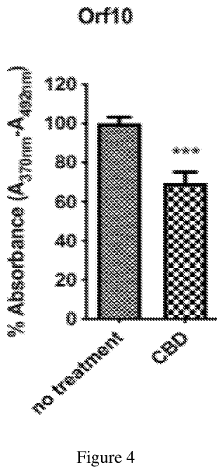

FIG. 4 provides a condition where HEK293 (human embryonic kidney) cells are transfected with plasmids expressing a vector expressing the viral Orf10 protein and further treated with Cannabidiol.

Surprisingly, significant reduction in mean BrdU incorporation was observed.

In cells expressing Orf10, BrdU incorporation was significantly reduced by treatment with any cannabinoid relative to untreated cells, except for delta 8-tetrahydrocannabivarin which exhibited less reduction. This could reflect a significant decrease in mean cell proliferation, or it could indicate that there are fewer cells, or a combination of these outcomes. Analysis is by 1-way ANOVA with Tukey's multiple comparison's test, where columns with different superscripts are significantly different, **P<0.01, ***P<0.001, ****P<0.0001.

In cells expressing Orf10 and treated with CBD, mean BrdU incorporation was 30.44% lower than in cells expressing Orf10 but untreated with cannabinoids.

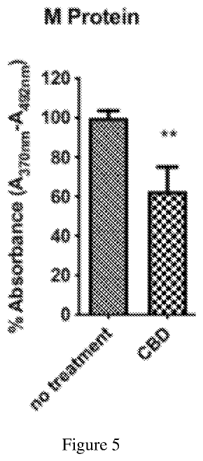

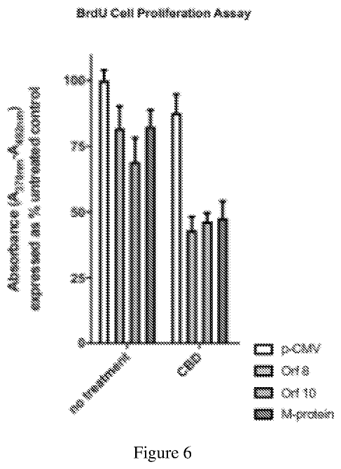

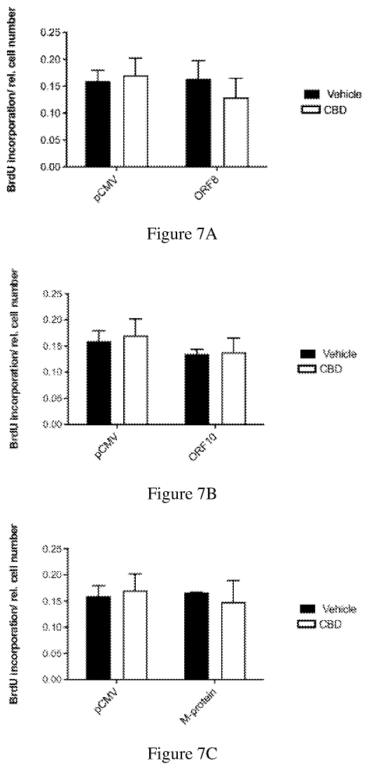

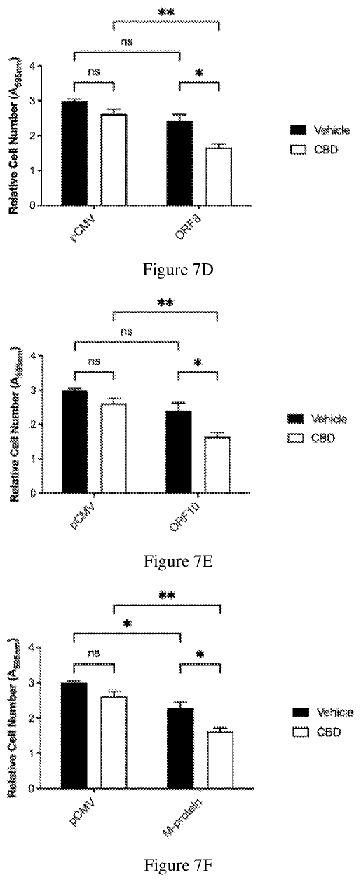

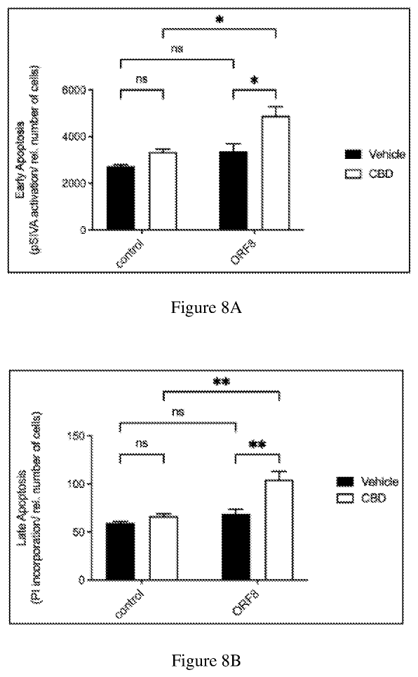

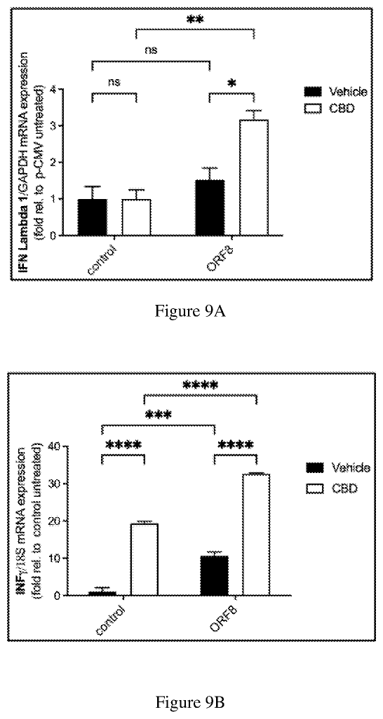

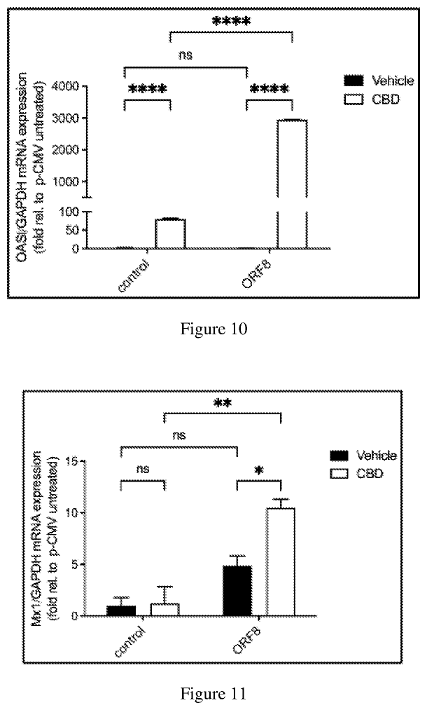

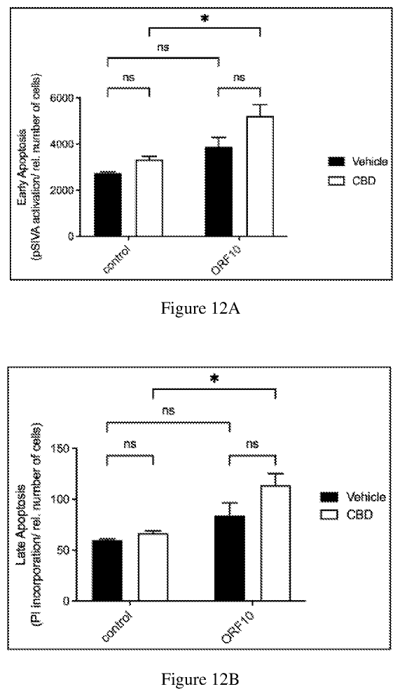

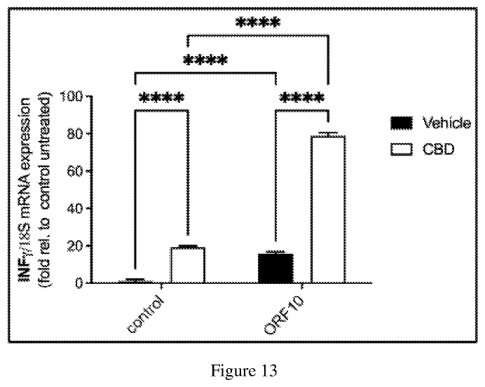

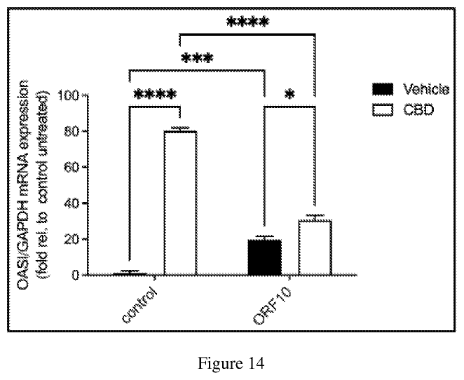

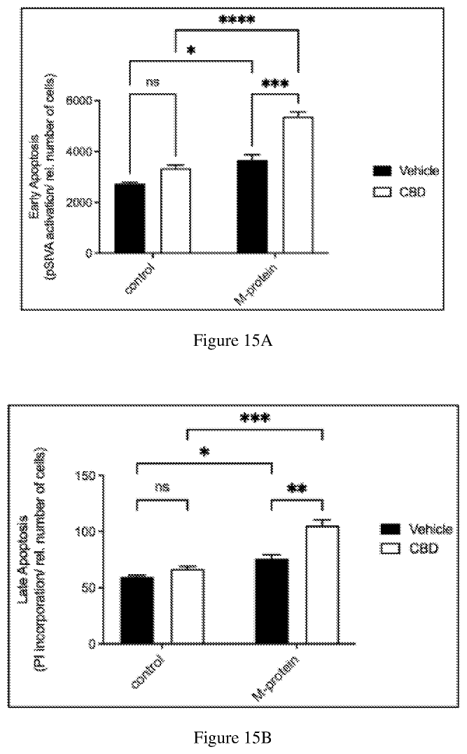

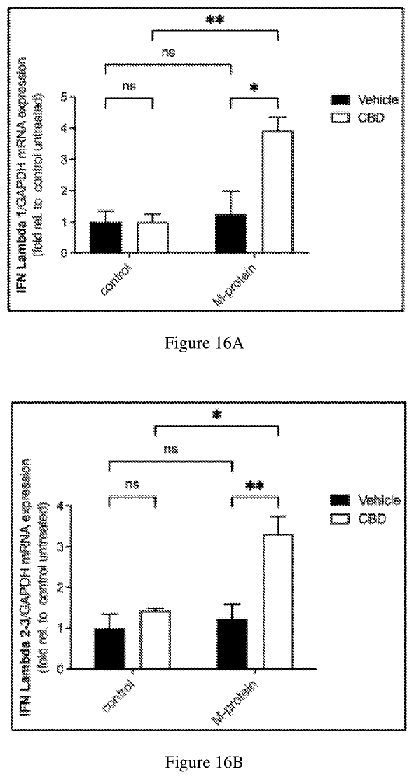

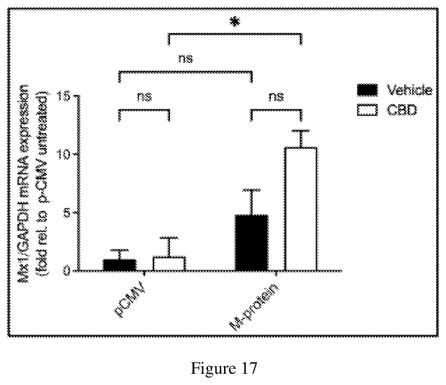

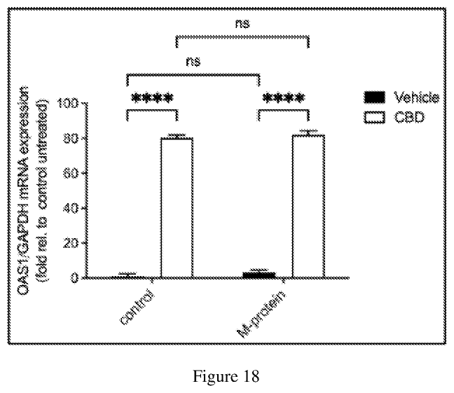

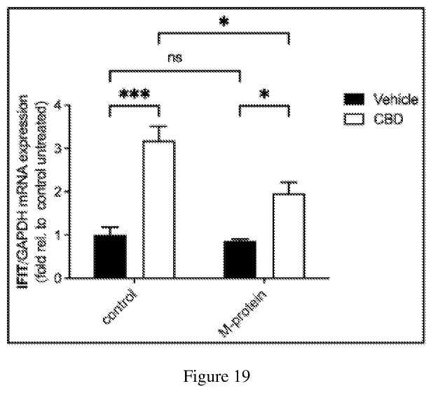

FIG. 5 provides a condition where HEK293 (human embryonic kidney) cells are transfected with plasmids expressing a vector expressing the viral M protein and further treated with Cannabidiol.

Surprisingly, significant reduction in mean BrdU incorporation was observed.

In cells expressing M protein, BrdU incorporation was significantly reduced by treatment with any cannabinoid relative to untreated cells.

This could reflect a significant decrease in mean cell proliferation, or it could reflect a decrease in the number of cells in each well proliferating at the same rate, or a combination of both. Analysis is 1-way ANOVA with Bonferonni's multiple comparison's test, where columns with different superscripts are significantly different, **P<0.01.

In cells expressing M protein and treated with CBD, mean BrdU incorporation was 37.28% lower than in cells expressing M protein but untreated with cannabinoids.

FIG. 6 combines data from all figures for ready comparison.

FIGS. 7A, 7B and 7C provide BrdU incorporation/cell proliferation, and therefore indicate the level of BrdU incorporation into nuclear DNA normalized to relative cell number in cells transfected with ORF8, ORF10 and M protein, respectively, and treated with or without Cannabidiol. These figures show that the level of BrdU incorporation per cell was not significantly different between cells transfected with control plasmid or with plasmids expressing ORF8 or ORF10 or M protein, whether treated with CBD or without (vehicle control). This indicates that the rate of HEK293 cell proliferation was not significantly altered by viral proteins or CBD, or a combination of both. It also indicated that in FIGS. 1 to 6, the decrease in BrdU incorporation was very likely due to a lower number of cells in each well, rather than a decrease in cell proliferation.

FIGS. 7D, 7E and 7F respectively provide an assay where adherent cells are stained by crystal violet and hence provide a measure of the relative cell number per well. These figures show that Cannabidiol does not significantly affect the relative number of cells per well when cells only express the control plasmid. FIG. 7D provides relative cell number when cells are transfected with either a control plasmid or plasmid transfected with ORF8 and treated with or without Cannabidiol.

This figure shows that expression of ORF8 without Cannabidiol treatment does not reduce relative cell numbers, but relative cell numbers are decreased when cells expressing ORF8 and are treated with Cannabidiol, both in comparison to cells expressing ORF8 but treated with vehicle only, or in comparison to cells transfected with control plasmid and treated with Cannabidiol. This shows that Cannabidiol combines with this SARS-COV-2 gene to cause a decrease in relative cell number that is only seen when the viral protein is combined with Cannabidiol.

FIG. 7E provides relative cell number when cells are transfected with either a control plasmid or plasmid transfected with ORF10 and treated with Cannabidiol.

This figure shows that expression of ORF10 without Cannabidiol treatment does not reduce relative cell numbers, but relative cell numbers are decreased when cells express ORF10 and are treated with Cannabidiol, both in comparison to cells expressing ORF10 but treated with vehicle only, or in comparison to cells transfected with control plasmid and treated with Cannabidiol. This shows that Cannabidiol combines with this SARS-COV-2 gene to cause a decrease in relative cell number that is only seen when the viral protein is combined with Cannabidiol.

FIG. 7F provides relative cell number when cells are transfected with either a control plasmid or plasmid transfected with M protein and treated with Cannabidiol. This figure shows that expression of Mprotein either with or without Cannabidiol will decrease relative cell numbers per well compared to cells transfected with the control plasmid alone and treated either with or without Cannabidiol, respectively.

However, in cells expressing M-protein, Cannabidiol treatment further enhanced the reduction in relative cell number.

FIGS. 8A and 8B provide respectively an early and late apoptosis data of HEK293 (human embryonic kidney) cells transfected with i) control plasmid expressing control vector and ii) plasmid expressing viral protein ORF8; and then treated with Cannabidiol. Cannabidiol treated cells which are transfected with control plasmid do not show any significant increase in early as well as late apoptosis but Cannabidiol treated cells which are transfected with plasmid expressing viral protein ORF8 have exhibited significant increases in early apoptosis and late apoptosis, both relative to ORF8-expressing cells treated only with vehicle control, and relative to control vector-expressing cells treated with Cannabidiol, indicating that Cannabidiol augments the cellular pro-apoptotic anti-viral response to ORF8, and this is specific to cells expressing ORF8.

FIG. 9A provides Interferon Lambda 1 mRNA levels produced when cells expressing ORF8 or control vector are treated with Cannabidiol compared to vehicle control.

In cells expressing ORF8, but not treated with CBD, Interferon Lambda 1 levels were not significantly elevated versus cells expressing only the empty-vector control plasmid. This highlights the problem that cells often have an inadequate innate anti-viral response to SARS-COV-2.

In cells expressing ORF8, CBD significantly increased expression of Interferon lambda 1 at 24 hours versus treatment with vehicle alone, indicating that CBD augments this anti-viral response to ORF8.

FIG. 9B provides that CBD augmented the expression of INF-gamma in both control and ORF8-expressing cells, but had a greater effect on this expression in ORF8 expressing cells.

FIG. 10 provides a highly significant increase in the expression of OAS1 (Oligoadenylate synthetases 1) gene in cells transfected with ORF8 protein and treated with Cannabidiol relative to all other groups and treatments.

FIG. 11 provides that Mx1 (Dynamin-Like GTPase myxovirus resistance protein 1) another interferon stimulated gene, is more highly expressed when cells transfected with ORF8 protein are treated with Cannabidiol, highlighting that Cannabidiol in combination with this SARS-COV-2 protein augments this anti-viral response.

FIGS. 12A and 12B respectively provide early apoptosis and late apoptosis data in cells transfected with a control plasmid or viral plasmid expressing ORF10 and treated with Cannabidiol. Cannabidiol induces apoptosis in cells transfected with ORF10 and treated with Cannabidiol to a significantly greater extent than in cells treated with Cannabidiol but expressing only control plasmid, indicating a specific ability of Cannabidiol to augment apoptosis when present in combination with the SARS-COV-2 ORF10 protein, but not when a non-viral plasmid is present.

FIG. 13 provides that in cells expressing ORF10, CBD significantly increased expression of Interferon gamma which is an indication of augmentation of the innate anti-viral response by cells. Expression of Interferon gamma is also seen in Cannabidiol treated cells transfected with a control plasmid, but to a lesser extent than in Cannabidiol-treated cells transfected with the SARS-COV-2 gene ORF10.

FIG. 14 provides expression of OAS1 in cells transfected with a control plasmid or plasmid expressing ORF10 and treated with Cannabidiol. Treatment with Cannabidiol significantly increased the induction of OAS1 in cells transfected with ORF10 or control plasmid relative to treatment with vehicle alone (i.e. without Cannabidiol).

FIGS. 15A and 15B respectively provide early apoptosis and late apoptosis data in cells transfected with a control plasmid or viral plasmid expressing M protein and treated with Cannabidiol. Cells transfected with M protein and treated with Cannabidiol significantly increased both early and late apoptosis compared to cells treated under the same conditions but transfected only with control plasmid. Cells transfected with M-protein and treated with Cannabidiol had significantly elevated early and late apoptosis also relative to cells expressing M protein but treated only with vehicle.

FIGS. 16A and 16B provide that Cannabidiol induced both INF-lambda 1 and INF-lambda 2/3 in cells expressing M-protein, indicating that CBD augments the interferon response to this SARS-COV-2 protein and augments this aspect of the innate intracellular anti-viral response.

FIG. 17 provides that cells transfected with both control plasmid and M protein and treated with cannabidiol have exhibited expression of Mx1. Cannabidiol induces Mx1 gene expression in cells transfected with M-protein and treated with Cannabidiol to a significantly greater extent than in cells treated with Cannabidiol but expressing only control plasmid.

FIG. 18 provides that the cells transfected with either control plasmid or M protein and treated with Cannabidiol have exhibited significantly greater expression of OAS1 gene compared to their respective vehicle-treated cells.

FIG. 19 provides that cannabidiol significantly increases expression of IFIT1 either in cells transfected with M protein or control plasmid, and therefore may help to prime the innate cellular immune system to enhance ability to launch an anti-viral defense.

### BACKGROUND OF THE ART

Viral proteins usually play critical roles in interfering with the host acquired immune response, but can also directly interfere with anti-viral innate immune responses mediated directly within infected cells that are meant to stop viral replication and spread. The pandemic of coronavirus disease 2019 (COVID-19) caused by the 2019 novel coronavirus (2019-nCOV or SARS-COV-2) infection has become a Public Health Emergency of International Concern (PHEIC). SARS-CoV-2 is highly pathogenic in human, having posed immeasurable public health challenges to the world.

SARS COV-2 is related to an earlier strain that also causes respiratory disease in humans, SARS COV. Prior characterization of SARS COV has facilitated decoding the SARS COV2 genome.

Genomic products of the SARS COV-2 genome are designated in lower case letters, in italics (e.g. orf10), while viral genes are designated in upper case letters (e.g. ORF10).

The pandemic of coronavirus disease 2019 (COVID-19) caused by the 2019 novel coronavirus (2019-nCOV or SARS-COV-2) infection has created havoc by infecting more than 127 million individuals across the world and by causing around 3 million deaths as of Mar. 29, 2021, with both the numbers of cases and deaths still climbing. It has been reported that some coronavirus proteins play an important role in modulating innate immunity of the host, but few studies have been conducted on SARS-COV-2.

Several independent research studies by Lu, R. et al.; Zhou, P. et al, Xu, J. et al., provided that SARS-COV-2 shares almost 80% of the genome with SARS-COV. Lu, R. et al further provided that almost all encoded proteins of SARS-COV-2 are homologous to SARS-COV proteins.

(SARS)-COV was identified as the etiologic agent of the 2002-3 international SARS outbreak. Chong-Shan Shi et al in a paper published in Journal of Immunology (2014) provides an insightful study on how SARS evades innate immune responses to cause human disease.

According to Shi, a protein encoded by SARS-COV designated as open reading frame-9b (ORF-9b) plays multiple roles as follows:
1. It localizes to mitochondria and causes mitochondrial elongation by triggering ubiquitination and proteasomal degradation of dynamin-like protein (DRP1), a host protein involved in mitochondrial fission;
2. It acts on mitochondria and targets the mitochondrial-associated adaptor molecule viz. Mitochondrial antiviral signaling protein (signalosome) (MAVS) by usurping poly(C)-binding protein 2 (PCBP2) and the HECT domain E3 ligase AIP4 to trigger the degradation of MAVS, TRAF3, and TRAF6. This severely limits host cell interferon responses.
3. Transient ORF-9b expression led to a strong induction of autophagy in cells. Shi reports as follows:

“These results indicate that SARS-COV ORF-9b manipulates host cell mitochondria and mitochondrial function to help evade host innate immunity. This study has uncovered an important clue to the pathogenesis of SARS-COV infection and illustrates the havoc that a small open reading frame can cause in cells.”

All viral proteins of SARS-COV-2 viz. NSP1, NSP2, NSP3, NSP4, NSP5, NSP6, NSP7, NSP8, NSP9, NSP10, NSP11, NSP12, NSP13, NSP14, NSP15, NSP16, S protein, ORF3a, E protein, M protein, ORF6, ORF7a, ORF7b, ORF8, N protein, ORF10 are being extensively researched for development of novel therapeutics to treat Covid-19 (Gordon, D. E et al, 2020).

Jin-Yan Li et al (2020) in their recently published “Short Communication” in Virus Research 286 (2020) 198074, screened the viral proteins of SARS-COV-2.

Li et al (2020) reports the following mechanism which is triggered upon viral infection.
i) Binding of several transcription factors, such as IRF-3 and NF-κB, to the interferon promoter to stimulate type I IFN (IFN-α/β) expression (García-Sastre and Biron, 2006) upon virus infection;
ii) Secretion of Interferon and its binding with its receptor;
iii) initiation of the JAK/STAT pathway and inducing the nucleus translocation of IFN-responsive transcriptional factors upon binding of interferon with its receptors;
iv) activation of genes containing interferon-stimulated response elements (ISREs) in their promoters, resulting in the expression of a set of IFN-stimulated genes (ISGs) establishing an antiviral state (Catanzaro et al., 2020).

Li further states that in response to this powerful selective environment, many viruses from diverse families, including filoviruses, poxviruses, influenza viruses, flaviviruses, and coronaviruses (CoVs), have evolved multiple passive and active mechanisms to avoid induction of the antiviral type I interferon, and they could optimize the intracellular resource for efficient virus replication (Volk et al., 2020).

Li et al found that the viral ORF6, ORF8 and nucleocapsid proteins were potential inhibitors of the type I interferon signaling pathway, a key component for antiviral response of host innate immunity. All the three proteins showed strong inhibition on type I interferon (IFN-β) and NF-κB-responsive promoters. Further examination by Li revealed that these proteins were able to inhibit the interferon-stimulated response element (ISRE) after infection with Sendai virus, while only ORF6 and ORF8 proteins were able to inhibit the ISRE after treatment with interferon beta.

SARS-COV-2 ORF6, ORF8, N and ORF3b are potent interferon antagonists, and in the early stages of SARS-COV-2 infection, delayed release of IFNs would hinder the host's antiviral response and then benefit virus replication. This, followed by the rapidly increased cytokines and chemokines attract inflammatory cells, such as neutrophils and monocytes, resulting in excessive immune infiltration causing tissue damage.

Khailany et al refers to an article by Koyama et al, 2020, wherein Koyama finds that ORF10, a short 38-residue peptide from SARS-COV-2 genome is not homologous with other proteins in the NCBI repository and Khailany further states that since ORF10 doesn't have any comparative proteins in the NCBI repository, it is one of a kind protein, which can be used to distinguish the infection more rapidly than PCR based strategies, but the further characterization of this protein is strongly required.

Interestingly, a paper available on chemrxiv.org by Seema Mishra titled “ORF10: Molecular insights into the contagious nature of pandemic novel coronavirus 2019-nCol” emphasized on the fact that ORF10 is an unknown protein with no homology to any known protein in organisms present till date. She further conducted immunoinformatics studies through which, it has been observed that among all ten 2019-nCOV proteins, ORF10 presents amongst the highest number of immunogenic, promiscuous CTL epitopes. (Cytotoxic T Lymphocytes).

While linking ORF10 to a contagious nature of pandemic novel coronavirus 2019-nCOV, she states as follows:

Through immunoinformatics studies, it has been observed that among all ten 2019-nCov proteins, ORF10 presents amongst the highest number of immunogenic, promiscuous CTL epitopes. These epitopes are part of a cluster with HTL epitopes, suggesting that there is a high degree of epitope conservation in ORF10. Conservation of protein sequence across organisms is not seen, and there is no known structural template on which to model and derive a structure to get structural and functional insights. Because there is altogether no conservation of its sequence, or structure, it may be presented as a novel protein to the immune system. Further, the human body may not have been able to utilize any memory B and T cells generated against other microorganisms to target ORF10 and fight this pathogen, contributing to its deadly, contagious nature.”.

Additionally, ORF8 protein is another protein that is not homologous with other proteins in the SARS COV genome (Xu, J. et al., Viruses 2020, 12, 244), although it does show very low homology to proteins encoded by other related viruses (Tang, X. et al. National Science Review 2020, 7, 1012-1023). The SARS COV-2 ORF8 protein is of a particular interest due to the recent finding that it is potential inhibitor of type I interferon signaling pathway, a key component for antiviral response of host innate immunity.

The gene for orf8 (Accession YP_009724396.1, UniProt ID PODTC8•NS8_SARS2), is encoded at the 3′ end of the SARS COV-2 genome. It results in a protein that is 121 amino acids long, with the N-terminal region forming a predicted signal peptide identifying a cleavage site at aa 15 (Target P-2.0 prediction). The predicted subcellular localization (using PSORTII, https://psort.hgc.jp/form2.html) is extracellular (55.6%).

However, over 80 cellular proteins that potentially interact with ORF8 (www.ebi.ac/uk/interact/interactors/id:P0DTC8) have been identified. These include mitochondrial proteins involved in metabolism, and cardiolipin and lipid synthesis (e.g. mitochondrial glutamate carrier 1, mitochondrial ATP synthase subunits alpha and beta, alpha trifunctional protein, and various dehydratases and enolases), Golgi proteins (e.g. Coatomer subunits α/β/,γ, etc), endoplasmic reticulum (ER) proteins (e.g. ER lectin 1, ER membrane protein complex subunit 1, etc), proteasomal proteins (e.g. 26S proteasome non-ATPase regulatory subunit 6, proteasome subunit alpha type-7), nuclear proteins (e.g. EIF3A, RBP2, etc.), and others.

ORF10 protein, being an unknown protein with no homology to any known protein in organisms present till date, and due to its unique association with SARS-COV-2, also serves an interesting candidate.

ORF10 (Accession YP_009725255.1, UniProt ID A0A663DJA2*), is predicted by PSORT II to likely be cytoplasmic (56.5% probability), but also mitochondrial (21.7%), nuclear (13%), secretory system vesicle-associated (4.3%) or ER-associated (4.3%). This viral protein is small, at only 38 amino acids, and has a predicted N-terminal transmembrane helix spanning amino acids 5-19.

Protein interaction data from the IntAct database (https://www.ebi.ac.uk/intact/interactors/id:A0A663DJA2*) indicates only 30 potential interactors. It is notable, however, that there are several common interactors between ORF8 protein and ORF10 protein, including mitochondrial, Golgi, and endoplasmic reticulum proteins.

The membrane glycoprotein (M protein, Accession YP_009724393.1)) is a structural protein that is highly conserved across all beta-coronaviruses, but has been found to have some sequence variants in the SARS COV-2 virus, with at least 7 amino acid substitutions identified thus far (M. Bianchi et al, BioMed Research International Vol 2020 Article ID 4389089). The M protein may be important for viral entry, replication, and particle assembly within host cells, as well as for viral budding. Data from an interaction study also suggests that M protein may interfere with mitochondrial metabolism (https://doi.org/10.1038/s41586-020-2286-9) and additional cellular processes.

There are a number of encoded non-structural proteins in the SARS COV-2 genome. The non-structural protein 5 (NSP5) is encoded in open reading frame 1a (orf1a) that produces a polypeptide (Accession #YP_009725295.1) and orf1ab (polypeptide Accession #YP_009724389.1), which are further processed to yield non-structural proteins including NSP5. A recent interaction study has suggested based on protein-protein interactions that NSP5, which is the main protease of the SARS COV-2 genome, may affect the ability of proteins to target the mitochondria and cause oxidative stress, and may be targeted therapeutically by anti-oxidant drugs, although this has not yet been shown experimentally.

A lack of basic knowledge about SARS COV-2 is a limiting factor for the development of novel therapeutics to treat this disease. Although SARS-COV-2 has been observed to share almost 80% of the genome with SARS-COV (Catanzaro 2020), given that there are differences in the infectivity, host interaction, and pathogenicity between these two viruses (2), ORF8 protein and ORF10 protein are of significant interest, as well as M protein and NSP5, among the other known individual proteins in the SARS COV-2 genome.

In recent times, interest in cannabidiol (CBD), which is a nonpsychoactive constituent of marijuana with potent antioxidant and anti-inflammatory effects, is rising exponentially.

CBD had been found to modulate translocation of various cellular proteins including transcription factors. CBD exposure rapidly increased TRPV2 protein expression and promoted its translocation to the cell surface of BV-2 cells (Samia Hassan 2014).

Chong-Shan Shi et al provides how a protein encoded by SARS-COV designated as open reading frame-9b (ORF-9b) localizes to mitochondria and causes mitochondrial elongation by triggering ubiquitination and proteasomal degradation of dynamin-like protein (DRP1), a host protein involved in mitochondrial fission (Shi et al 2014). CBD has been found to rescue levels of dynamin 1 that are reduced in iron-overloaded cells (da Silva V K et al 2014).

Enkui Hao et al reports protective effects of CBD against doxycycline-induced cardiotoxicity and cardiac dysfunction by
(i) attenuating oxidative and nitrative stress, (ii) improving mitochondrial function,
(iii) enhancing mitochondrial biogenesis, (iv) decreasing cell death and expression of MMPs and (v) decreasing myocardial inflammation.

CBD had been found to modulate translocation of various cellular proteins including transcription factors (Huang Y et al, 2019) and membrane cation channels (Hassan S et al, 2014).

CBD has been found to function in the modulation of mitochondrial calcium metabolism, mitochondrially-mediated apoptosis, mitochondrial ferritin regulation, the electron transport chain, and mitochondrial biogenesis and fission (da Silva V K, 2018; Hao E et al, 2015; McKallip R J et al, 2006; Ryan D et al, 2009 and Valvassori S S et al, 2013).

In the inhouse work on adenovirus (unpublished data), inventors have found lower complex I activity in infected cells. However, it has been shown that CBD treatment of rats increased complex I, II, III, and IV activity, likely due to enhanced accumulation of calcium inside the mitochondria, which increased the activity of calcium-sensitive dehydrogenases and promoted availability of NADH for oxidative phosphorylation (Valvassori S S et al, 2013).

Various researchers have shown that CBD shows significant promise for the treatment of numerous cancers, based primarily on evidence of induction of a pro-apoptotic effect (Jeong S et al, 2019; Jeong S, Yun H K et al, 2019; Sultan A S et al, 2018). In inhouse work, inventors have found that in metabolically dysregulated cells, CBD decreases cell death, and has no effect in normal cells (unpublished data). The same is also observed by Oláh A et al, 2016; and Solinas M et al, 2012. Cell type may also be a factor in determining response. In an in vivo model of hypoxic-ischemic injury, mouse forebrain tissues subjected to oxygen-glucose deprivation had a 5-fold increase in caspase 9 activation that was attenuated nearly 50% by 100 μM CBD (Castillo A et al, 2010), while 5 μM CBD also significantly attenuated apoptosis and oxidative stress in cultured HT22 hippocampal neurons subjected to oxygen-glucose deprivation (Sun S et al, 2017).

Whether CBD, or other cannabinoids, can attenuate potential pro-apoptotic effects of viral proteins requires direct investigation.

Following data is found reported on the modulation of lipid metabolism by CBD:
a. CBD is reported to stimulate sphingomyelin hydrolysis in cells cultured from a patient with Niemann-Pick disease, suggesting that it may help to relieve symptoms caused by accumulation (Burstein S et al, 1984).
b. In a paper published almost 40 years ago, it was also found that cannabidiol and other cannabinoids dose-dependently inhibited cholesterol esterification in cultured human fibroblasts, without affecting triacylglycerol or phospholipid synthesis (Cornicelli J A, et al 1981).
c. CBD treatment of cultured mouse microglial cells also alters the accumulation of specific species of N-acylethanolamines (N-AE) in membrane lipid rafts (Rimmerman N et al, 2012). Although minor components, N-AE are highly bioactive. As docking sites for membrane-bound proteins, and ‘scaffolding sites’ for the assembly of signaling complexes, lipid rafts are important sites in cells for physiological and pathophysiological regulation.
d. Regulation of lipid rafts may also have specific importance in COVID-19. The ACE2 receptor, which binds the Spike protein of SARS COV-2 to initiate cellular entry and infection, is located within cholesterol-rich lipid domains (Lu Y et al, 2008).

Since both sphingomyelin and free (i.e. unesterified) cholesterol are significant components of lipid rafts, these reports indicate a potential role for CBD in regulation of these membrane subdomains.

CANNABIDIOL (CANNABIDIOL) is the main cannabinoid constituent of Cannabis sativa plant. It binds very weakly to CB1 and CB2 receptors.

CANNABIDIOL does not induce psychoactive or cognitive effects and is well tolerated without side effects in humans, thus making it a putative therapeutic target. In the United States, the CANNABIDIOL drug Epidiolex was approved by the Food and Drug Administration in 2018 for the treatment of two epilepsy disorders: Dravet Syndrome and Lennox/Gasteaut Syndrome.

CANNABIDIOL is designated chemically as 2-[(1R,6R)-3-Methyl-6-(1-methylethenyl)-2-cyclohexen-1-yl]-5-pentyl-1,3-benzenediol. The chemical structure is as follows.

The U.S. Pat. No. 6,410,588 discloses the use of CANNABIDIOL to treat inflammatory diseases.

The PCT publication no. WO2001095899A2 relates to CANNABIDIOL derivatives and to pharmaceutical compositions comprising CANNABIDIOL derivatives being anti-inflammatory agents having analgesic, antianxiety, anticonvulsive, neuroprotective, antipsychotic and anticancer activity.

CANNABIDIOL (CANNABIDIOL(CANNABIDIOL)) is approved as an anti-seizure drug (Barnes, 2006; Devinsky et al., 2017). CANNABIDIOL lacks adverse cardiac toxicity and ameliorates diabetes/high glucose induced deleterious cardiomyopathy (Cunha et al., 1980; Izzo, Borrelli, Capasso, Di Marzo & Mechoulam, 2009; Rajesh et al., 2010).

### DETAILED DESCRIPTION OF THE INVENTION

### Definition of Terms

The terms Cannabidiol and CBD are used synonymously.

The term early apoptosis covers early apoptosis as a stage of apoptosis.

Apoptosis early after infection indicates timepoint when the cells undergo apoptosis after infection. This covers both early and late apoptosis as long as they happen early after infection. In the present invention it is noted that cells undergo apoptosis at 24 hrs which indicates that Cannabidiol causes Apoptosis early after infection wherein some cells may be in early apoptosis and some cells may be in late stage of apoptosis.

Cannabidiol has been shown to be effective in protecting endothelial function and integrity in human coronary artery endothelial cells (HCAECs) by Rajesh M et al. They have proposed following action of Cannabidiol by inhibiting
Reactive oxygen species production by mitochondria;
NF-κB activation;
Transendothelial migration of monocytes;
Monocyte-endothelial adhesion in HCAECs.

Nagarkatti et al provides as follows:
Cannabinoids, the active components of Cannabis sativa, and endogenous cannabinoids mediate their effects through activation of specific cannabinoid receptors known as cannabinoid receptor 1 and 2 (CB1 and CB2).
The cannabinoid system has been shown both in vivo and in vitro to be involved in regulating the immune system through its immunomodulatory properties.
Cannabinoids suppress inflammatory response and subsequently attenuate disease symptoms. This property of cannabinoids is mediated through multiple pathways such as induction of apoptosis in activated immune cells, suppression of cytokines and chemokines at inflammatory sites and upregulation of FoxP3 regulatory T cells.
Cannabinoids have been tested in several experimental models of autoimmune disorders such as multiple sclerosis, rheumatoid arthritis, colitis and hepatitis and have been shown to protect the host from the pathogenesis through induction of multiple anti-inflammatory pathways.

Vuolo et al has demonstrated Role of Cannabidiol Treatment in Animal Model of Asthma. The levels of all 6 cytokines implicated in asthma viz. TNFα, IL-6, IL-4, IL-13, IL-10, and IL-5 were determined in Control animals, asthma induced animals and asthma induced animals treated with Cannabidiol. Induced asthma has increased all 6 cytokines; however, in animal group treated with CBD, levels of all cytokines has been reduced significantly. This action of Cannabidiol is very important not only in asthma but in other conditions where a rise in cytokines is reported. Recent studies by Huang, C. et al. have shown that in addition to dyspnea, hypoxemia, and acute respiratory distress, lymphopenia, and cytokine release syndrome are also important clinical features in patients with severe SARS-COV-2 infection. Thus, Cannabidiol is also proposed as a treatment for Covid-19 due to its ability to reduce cytokines.

The inventors of the instant invention propose that through multiple known and unknown effects, Cannabidiol will serve as a highly desired therapeutic agent. It is a cardioprotective drug and role of Cannabidiol in reducing LQT is of vital significance.

Cannabidiol has been reported previously to be effective in protecting endothelial function and integrity in human coronary artery endothelial cells (HCAECs). Cannabidiol has reduced cytokines in induced asthma. Cannabidiol plays multiple roles such as anti-inflammatory, inhibitor of cytokines, agent to reduce LQT and a cardioprotective agent.

Long term treatment with Cannabidiol has been considered safe.

The invention also covers method of treating individuals suffering from any heart condition, any respiratory condition or any infection or condition where rise of cytokine and/or inflammation is observed by administering Cannabidiol compositions alone or with another suitable therapeutic.

These compositions may be consumed in the mono-treatment with Cannabidiol or as a prophylactic or as an adjunct therapy taking the help of a calendar-pack blister card encompassing Cannabidiol tablets along with the afore-mentioned.

The invention also covers administering Cannabidiol compositions prophylactically under certain circumstances so that any treatment which may be required in immediate future shall not cause LQT, cytokine elevation, inflammation and injury to heart.

The instant invention provides compositions and methods to enhance safety profile of any treatment including an antiviral treatment particularly including treatment for Covid-19 wherein the treatment includes administration of one or more drugs that may cause drug induced LQTs.

Cannabidiol would produce beneficial effects in one or more of the following pathogenesis of various disorders including, but not limited to, COVID-19, SARS, MERS, influenza, acquired, induced and drug related long QT syndrome, long QTc syndrome, long QRS syndrome, cardiomyopathy, heart failure, arrhythmia, myocardial ischemia, myocardial infarction (MI), arrhythmias of ischemic and non-ischemic origin, inflammation, vascular dysfunction, cardiomyopathy, cardiac remodelling, maladaptation, anginas of different types, drug induced heart failure, cardiac injury, iatrogenic heart and vascular diseases, or any combination thereof.

The inventors have previously proposed that Role of Cannabidiol in treating Covid-19 would be multifold. Cannabidiol is considered safe for chronic use and is cardioprotective in nature. It can reduce cytokines and act as anti-inflammatory agent. Most importantly, it reduces LQT and can prevent/rescue hyperexcitability of cardiac ion channels. In this way, it can enhance safety profile of the treatment which recommends administration of drugs for treating Covid-19 but capable of causing LQT and enables masses to receive best possible treatment.

All viral proteins of SARS-COV-2 viz. NSP1, NSP2, NSP3, NSP4, NSP5, NSP6, NSP7, NSP8, NSP9, NSP10, NSP11, NSP12, NSP13, NSP14, NSP15, NSP16, S protein, ORF3a, E protein, M protein, ORF6, ORF7a, ORF7b, ORF8, N protein, ORF10 are being extensively researched for development of novel therapeutics to treat Covid-19 (Gordon, D. E et al, 2020).

Understanding the cellular properties and function of viral proteins will allow for testing of new therapies and strategic interventions for COVID-19. The present inventors have initiated work to elucidate mechanism of action of ORF8, ORF10, M protein and NSP5). These novel proteins, ORF8 and ORF10, have not yet been fully characterized experimentally, and their functions in cells cannot be inferred from prior work. The functions of the SARS COV-2 form of M protein, and NSP5 are poorly understood. Indeed, little is known yet about the proteins that make up the SARS COV-2 genome, since they all contain differences compared to other known viral proteins. Knowledge of the cellular function and pathophysiological roles of these novel proteins in the SARS COV-2 genome is expected to provide potential new targets for therapeutic intervention. Further, the work is initiated on certain compounds which may interfere with the action of these viral proteins and prove to be useful to mankind to fight the pandemic. These compounds are those which may particularly reverse cellular perturbations caused by these viral proteins.

As on Mar. 26, 2021, the worldometers reports 126,203,749 cases of Covid-19 worldwide and 2,769,596 deaths.

Initial infection with a virus does not cause severe disease (the estimated infectious dose is 1000 virions). Illness only occurs when the virus enters cells and hijacks the cellular machinery to replicate, forming 1000s or millions of new virions.

SARS-COV-2 has many variants, but some are of particular importance due to
i) increased transmissibility,
ii) increased virulence, and
iii) reduced effectiveness of vaccines.

Notable Variants of SARS-COV-2 include

Cluster 5, Lineage B.1.1.7, Lineage B.1.1.207, Lineage B.1.1.317, Lineage B.1.1.318, Lineage B.1.351, Lineage B.1.429/CAL.20C, Lineage B.1.525

### Lineage P.1, Lineage P.3.

There are many mechanisms to attack virus. Most methods focus on inhibiting viral replication alone. One unique way to prevent the virus from replicating initially and then spreading and also from mutating, is making unavailable to the virus the infected host machinery. If the innate immune response of infected cells causes an early apoptosis causing death of infected cells, this will fragment the infected cellular machinery of the host, preventing i) replication and ii) the formation of new, infectious virions that can spread throughout the body, causing an overwhelming infection.

One problem with COVID-19 is that the innate immune response is often inadequate. Viral entry triggers interferons but if interferons are not triggered in sufficient amount then such inadequate induction of interferons is problematic.

Researchers have been studying prevention of viral replication through one or more means such as for example by measuring reduction in number of viral RNA after treatment with drugs by comparing viral RNA number in drug-treated cells and viral RNA content in vehicle-treated controls. In other studies, cells treated with drug and subjected to infection are stained for spike proteins and % of cells expressing spike proteins are plotted.

The present inventors focused on three viral proteins viz. ORF8, ORF10 and M protein.

ORF8 is an accessory protein that has been proposed to interfere with immune responses of host. ORF8 is unique in that it maybe dispensible in viral replication but it has a unique role of evading immune surveillance of host cells i.e. it has a role in the way virus evades immunity of host cell.

Khailany et al refers to an article by Koyama et al, 2020, wherein Koyama finds that ORF10, a short 38-residue peptide from SARS-COV-2 genome is not homologous with other proteins in the NCBI repository and Khailany further states that since ORF10 doesn't have any comparative proteins in the NCBI repository, it is one of a kind protein, which can be used to distinguish the infection more rapidly than PCR based strategies, but the further characterization of this protein is strongly required.

The membrane glycoprotein (M protein, Accession YP_009724393.1)) is a structural protein that is highly conserved across all beta-coronaviruses but has been found to have some sequence variants in the SARS COV-2 virus, with at least 7 amino acid substitutions identified thus far (M. Bianchi et al, BioMed Research International Vol 2020 Article ID 4389089). The M protein may be important for viral entry, replication, and particle assembly within host cells, as well as for viral budding. Data from an interaction study also suggests that M protein may interfere with mitochondrial metabolism (https://doi.org/10.1038/s41586-020-2286-9) and additional cellular processes.

In an earlier filed co-pending application IN202021030633 the inventors pondered upon a question whether CBD, or other cannabinoids, can attenuate potential pro-apoptotic effects of viral proteins and concluded that the question cannot be answered unless direct investigation is done. First, whether viral proteins exhibit pro-apoptotic effects or not should be established and then whether cannabinoids alter the effect of proteins needs investigation.

When host cells are infected with virus, they transcribe interferons that will block RNA processing, to try to block viral replication. Viruses ‘hijack’ the cellular machinery to make copies of themselves, which requires RNA processing. Interferons are made as a very early response, when a virus particle enters a cell, and they shut down RNA-mediated processes in cells. This stops viruses from replicating. However, this action of interferon may also stop cells from dividing, and can cause them to undergo apoptosis, and die. However, most of the times cells selectively block viral proteins while allowing cellular proteins to continue being made.

Thus, it is essential to find factors that can augment the initial intracellular anti-viral defenses of cells, particularly those defenses which host cells can launch immediately upon viral entry such as restoring type I, II or III interferon signaling pathways.

The present invention provides pharmaceutical compositions and methods for treating Covid-19 infectious disease comprising administering to patient such pharmaceutical compositions comprising therapeutically effective amount of Cannabidiol wherein such administration of said pharmaceutical composition to the said patient produces an enhancement/augmentation of innate immunity of the patient due to at least one of the following effects,
i) infected patient cells undergo apoptosis early after infection;
ii) induction of interferon transcription in the patient;
iii) induction of interferon-induced antiviral effectors in the patient.

When infected patient cells undergo apoptosis it renders them not available to the virus for mutation.

Thus, the present invention provides compositions and methods of preventing or reducing mutation of Sars-Cov-2 virus in a patient wherein said method comprises administering a pharmaceutical composition comprising therapeutically effective amount of cannabidiol to a patient suffering from Covid-19 by causing infected patient cells to undergo apoptosis early after infection which renders them not available to the virus for mutation.

The present invention also provides pharmaceutical compositions and methods for prophylaxis or prophylactic treatment of Covid-19 infectious disease comprising administering to mammal/human such pharmaceutical compositions comprising therapeutically effective amount of Cannabidiol wherein such administration of said pharmaceutical composition to the said mammal/human produces an enhancement/augmentation of innate immunity of the patient due to at least one of the following effects,
i) induction of interferon transcription in the patient;
ii) induction of interferon-induced antiviral effectors in the patient.

When compositions of the present invention are used for prophylaxis or prophylactic treatment of the Covid-19 infectious disease, it has been surprisingly found that no cells of such mammal/human who has not been infected undergo any apoptosis showing that these methods and compositions ‘prime’ the system of mammal/human to respond to virus, but don't alone cause the system to start to die.

Cannabidiol compositions and methods thus prepare even heathy individual for threat of the virus without causing harm to any cells. In such mammals/humans induction of interferons or induction of interferon induced anti-viral effectors are observed which also surprisingly do not cause apoptosis of uninfected/healthy cells.

The interferons most commonly induced in both cases i.e while treating patients and while prophylactically treating mammals/human not infected by the virus include include Type II and Type III interferons.

The most common interferon induced antiviral effectors found in both cases i.e while treating patients and while prophylactically treating mammals/human not infected by the virus include OAS1, Mx1, IFIT1 genes.

The present invention also provides compositions comprising therapeutically effective amount of Cannabidiol and methods for mammals and humans who are about to encounter virus or about to get infected due to various reasons. These humans are sometimes at higher risk because they are from a region where wave of pandemic is stronger. They are at higher risk because they are front line workers or health workers or have co-morbidities or they are quarantined because they have come in contact with patient, or they are frequent travelers. This category also includes mammals and humans who are not at higher risk but they are still about to get infected. It can be concluded that treatment with Cannabidiol in absence of virus (as shown from data where HEK293 (human embryonic kidney) cells are transfected with a control plasmid expressing control vector and treated with Cannabidiol also induce interferons and interferon induced antiviral effector such as OAS1, Mx1 and IFIT1) induces interferons and anti-viral effectors without actually triggering apoptosis. This readiness increases the likelihood that someone exposed to virus would trigger apoptosis right away in infected cells, preventing replication and spread of the infection (and therefore preventing illness).

Initial infection with a virus does not cause severe disease (the estimated infectious dose is 1000 virions). Illness only occurs when the virus enters cells and hijacks the cellular machinery to replicate, forming 1000s or millions of new virions.

However, when someone who is about to encounter virus or about to get infected takes Cannabidiol, it would raise the ‘infectious dose’ of the virus that would be needed to cause disease. In such condition, virus can't take hold, replicate sufficiently, and make someone sick.

The host cells are primed by induction of interferons and higher transcription of interferon induced anti-viral effector such as OAS1, so that they are better prepared to undergo apoptosis once they also encounter virus (but not harmful in the absence of virus). This indicates a potential that CBD ‘primes’ cells to be ready to respond to a viral threat, upon expression of viral genes.

Therefore, the present invention provides a pharmaceutical compositions and methods for administering compositions comprising therapeutically effective amount of Cannabidiol for use in preventing or better preparing for Covid-19 infectious disease in mammals/humans who are about to get infected Covid-19 infectious disease wherein administration of said pharmaceutical composition to the mammal/human produces an enhancement/augmentation of innate immunity in such mammal/human due to at least one of the following effects,
i) induction of interferon transcription in the mammal/human;
iii) induction of interferon-induced antiviral effectors in the mammal/human;

wherein such induction is not associated with apoptosis of cells in the beginning enabling cells to get primed/prepared for viral threat and wherein such cells are better able to prepare for infection including increase in the infectious dose of virions to the organism than usual dose and wherein the cells undergo apoptosis early after infection which renders the cells not available to the virus for replication and/or mutation.

The present invention includes a number of experiments carried out using a plasmid transfected with viral proteins.

HEK293 (human embryonic kidney) cells were chosen for transfecting with various viral proteins. HEK293 were seeded in 96 well plates, then transfected with plasmids expressing an empty control vector (pCMV-3Tag-3a) or vectors expressing the viral Orf8, Orf10 or M proteins. A few hours later the cells were treated with 1 μM of the cannabinoid, then grown for 24 hours, and assayed using a colorimetric ELISA that detects BrdU incorporation.

The studies undertaken by the present inventors focused on following experiments where BrdU is measured where control plasmid and plasmid expressing viral proteins are treated with cannabidiol,
i) to study effect of cannabidiol on nuclear BrdU incorporation in cells transfected with control plasmid expressing a control vector;
ii) to study effects of cannabidiol on nuclear BrdU incorporation in cells transfected with plasmid expressing viral proteins;

BrdU is incorporated into the nucleus of dividing cells, and therefore can provide a relative measure of cell proliferation. However, since this measure is dependent on the number of cells present, measures of BrdU incorporation can only be interpreted to indicate changes in cell proliferation rate after the data are normalized to the relative number of cells that are present and being measured. Thus, a decrease in BrdU incorporation could mean that cell proliferation rates are lower, or it could mean that cell proliferation rates are not different, but that there are fewer cells being measured.

Effects of Viral proteins ORF8, ORF10, and M protein are determined on BrdU incorporation into HEK293 (human embryonic kidney) cells and further investigation is conducted to check whether treatment of transfected cells with Cannabidiol reverses any observed effect of viral protein.

It was surprisingly observed that Viral proteins did not have much impact on BrdU incorporation of HEK293 (human embryonic kidney) cells. Although no significant impact was observed, a slight reduction in BrdU incorporation rates of HEK293 (human embryonic kidney) cells was observed with all viral proteins where reduction was higher than that of a reduction where a control plasmid expressing a control vector was used. For HEK293 (human embryonic kidney) cells, even a control plasmid expressing a control vector was a foreign body, but no significant reduction in BrdU incorporation due to the control plasmid was observed.

It was further surprisingly observed that in HEK293 (human embryonic kidney) cells transfected with various viral proteins of SARS-COV-2 when treated with Cannabidiol, a sharp and significant reduction in BrdU incorporation rates of these cells were observed. This effect was common for all viral genes tested.

The inventors have run 2-way ANOVA. This has been tested in multiple separate assays on different days/weeks, with n=5 to 6 biological replicates (where separate passages of cells were considered different biological replicates). Each biological replicate was seeded in 2 to 6 technical replicates per plate, and those were averaged at each trial to give n=1 for that biological replicate in that trial. Note that these data were not normalized to the relative number of cells that were present in each well.

These results are presented in FIGS. 1-5 and table 1 below:

TABLE 1
Studies conducted for measuring BrdU incorporation levels

| Transfection with | | | | |
|---|---|---|---|---|
| control plasmid or | | | | |
| plasmid with viral | | | | |
| protein [symbol] | | | | |
| Treatment or no | | | | |
| treatment with | | | | |
| Cannabidiol [symbol] | p-CMV | ORF8 | ORF10 | M-protein |
| No treatment | Trans- | Trans- | Trans- | Trans- |
| with | fection | fection | fection | fection |
| Cannabidiol | with | by plasmid | by plasmid | by plasmid |
| [symbol] | control | expressing | expressing | expressing |
| | plasmid | ORF8 | ORF10 | M-protein |
| | FIG. 1 | FIG. 1 | FIG. 1 | FIG. 1 |
| Treatment | Trans- | Trans- | Trans- | Trans- |
| with | fection | fection | fection | fection |
| Cannabidiol | with | by plasmid | by plasmid | by plasmid |
| [symbol] | control | expressing | expressing | expressing |
| | plasmid + | ORF8 + | ORF10 + | M protein + |
| | treatment | treatment | treatment | treatment |
| | with CBD | with CBD | with CBD | with CBD |
| | FIG. 2 | FIG. 3 | FIG. 4 | FIG. 5 |

The BrdU incorporation level into DNA was measured by incorporating and quantifying bromodeoxyuridine (BrdU) into DNA of actively proliferating cells. The absorbance values are measured by ELISA assay with a BioTek Synergy H1 Hybrid Multi-Mode Microplate reader assay at 370 nm (reference wavelength: approx. 492 nm). FIGS. 1-5 provide results of the tests performed. FIG. 6 combines data from all figures for ready comparison. The absorbance is expressed as % untreated control and the absorbance values are normalized.

The absorbance value reflects the average quantity ofBrdU incorporated into nuclei of cells in each well of cells, as DNA of these cells have incorporated bromodeoxyuridine which is measured in the assay. The absorbance when cells are transfected with a plasmid expressing an empty control vector (pCMV-3Tag-3a) is taken as a control. pCMV-3Tag-3A is a control vector that expresses a very small protein comprised of 3 FLAG tags in tandem (the amino acid sequence is DYKDDDDKDYKDDDDKDYKDDDDK (SEQ ID No: 15). All absorbance values are to be compared to the control value. A significant deflection from the control value should reflect either reduction or enhancement in BrdU incorporation. It could reflect a difference in cell proliferation, or it could reflect a difference in cell number, indicating enhancement of cell apoptosis, which would reduce the number of cells per well.

The viral infected cells are simulated by transfecting cells with a plasmid expressing different viral proteins. Transfected cells are grown for 24 hrs in order to allow time for viral protein expression before they are assayed using a colorimetric ELISA that detects BrdU incorporation. No significant deflection but a slight reduction is observed in absorbance values, which indicates that viral proteins alone have no, or only a very small inhibitory effect on BrdU incorporation.

The effects of Cannabidiol on viral infected cells are simulated by treating cells transfected with a plasmid expressing different viral proteins with Cannabidiol. Transfected cells are grown for 24 hrs in order to allow time for viral protein expression before they are assayed using a colorimetric ELISA that detects BrdU incorporation. Surprisingly, after treatment with cannabinoids, significant reductions in absorbance are observed.

Since the absorbance when cells are transfected with a plasmid expressing an empty control vector is observed to be maximum, this absorbance value is normalized to 100% or 100 and all other values are plotted relative to the 100%.

As provided in FIG. 3, in cells expressing ORF8 protein and treated with CBD, mean BrdU incorporation was 37.28% lower than in cells expressing ORF8 protein but untreated with cannabinoids.

As provided in FIG. 4, in cells expressing Orf10 and treated with Cannabidiol, mean cell BrdU incorporation was 30.44% lower than in cells expressing Orf10 but untreated with cannabinoids.

As provided in FIG. 5, in cells expressing M protein and treated with CBD, mean cell BrdU incorporation was 37.28% lower than in cells expressing M protein but untreated with cannabinoids.

Thus, Cannabidiol impacted BrdU incorporation levels of HEK293 (human embryonic kidney) cells transfected with all three viral proteins of SARS-COV-2. There is a significant reduction in each case. Surprisingly, Cannabidiol in untreated cells as well as in cells transfected with a control plasmid expressing a control vector does not reduce cell proliferation. This is an indication of tremendous potential of Cannabidiol in impacting either cell number or cell proliferation in infected cells.

Reduction in absorbance/reduction in BrdU incorporation after treatment with Cannabidiol does reflect a few things.

1. Interferons are produced as a response to entry of virus. However, interferons stop cell proliferation and increase apoptosis, which would reduce cell numbers and also reduce BrdU incorporation. Therefore, even when interferons are produced, it is likely to cause a reduction in absorbance. Thus, reduction in absorbance may reflect an enhanced production of interferons, and an increase in the innate intracellular response to these SARS-COV-2 genes.

2. Reduction in BrdU incorporation and therefore absorbance can also be due to increased cell apoptosis. If reduction in absorbance is due to lower cell numbers due to increased cell apoptosis, it also reflects activation of innate cellular defense to viral genes. It is an indication that transfected cells (cells transfected with viral genes) are undergoing programmed cell death. This process has a potential where infected host cells can selectively undergo apoptosis leaving behind healthy cells. Thus, host machinery of infected cells are destroyed or fragmented and virus has no place for replication or mutation, suppressing the creation of new variants.

3. It is possible that interferons are also produced and transfected cells are also undergoing apoptosis.

In each case, activation of cell defense after treatment with Cannabidiol is apparent.

Recently, Banerjee et al have reported that all of the viral proteins of SARS-COV-2 responsible for inhibiting RNA processing by cells (NSP1, NSP8, NSP9, and NSP16) are produced in the first stage of the viral life cycle, prior to generation of double stranded RNA (dsRNA). dsRNA is detected by host immune sensors and a type I interferon response is triggered. This means that SARS-COV-2 viral proteins that can stop the transcription of interferons are formed earlier than the events that trigger a type I interferon response. Therefore, unless a mechanism can allow interferons to still be produced, in the face of interferon production arrest by SARS CoV-2 proteins, then cell defenses to viral infection cannot be activated.

The present study provides such an early defense mechanism, where cells due to presence of Cannabidiol either rapidly produce interferons upon viral protein expression at viral entry or cause apoptosis of infected cells as a result of cellular defense.

The data from FIGS. 1-6 reflecting reduction in BrdU incorporation were not normalized to cell number. This may mean that reduction in BrdU incorporation as noted when cells transfected with a control plasmid and plasmid transfected with different viral proteins and treated with cannabidiol is actually not due to a reduction in the rate of cell proliferation, but to a reduction in cell number due to increased apoptosis. To confirm whether cell proliferation is reduced by treatment with Cannabidiol, cell proliferation data should be normalized to cell number. FIGS. 7A, 7B and 7C provide BrdU incorporation/cell proliferation, where the measure of the relative incorporation of BrdU is normalized to relative cell number per well, for cells transfected with ORF8, ORF10 and M protein respectively and treated with Cannabidiol. It also provides cells transfected with a control plasmid and treated with Cannabidiol. It is noted that there is no significant difference in BrdU incorporation/cell proliferation rate when it is normalized to cell number, i.e. there is no reduction in cell proliferation rate when cells transfected with a control plasmid or with viral protein are treated with Cannabidiol. This means that treatment of cells transfected with a control plasmid or plasmid expressing viral proteins with Cannabidiol does not reduce cell proliferation rate, although reduced BrdU incorporation level was observed earlier. It is further necessary to find reasons for the earlier observed BrdU incorporation reduction. This can be done by crystal violet staining assay, which provides a relative measure of the number of adherent cells present in a well. FIGS. 7D, 7E and 7F respectively provide crystal violet assay where cells are stained by crystal violet and hence provide relative cell number. FIG. 7D provides relative cell number when cells are transfected with either a control plasmid or plasmid expressing ORF8 and treated with Cannabidiol. FIG. 7E provides relative cell number when cells are transfected with either a control plasmid or plasmid expressing ORF10 and treated with Cannabidiol. FIG. 7F provides relative cell number when cells are transfected with either a control plasmid or plasmid expressing M protein and treated with Cannabidiol.

Crystal violet assay provides that for cells transfected with each viral protein and treated with Cannabidiol, there is a significant reduction in relative cell number. This signals at apoptosis of cells treated with Cannabidiol and necessitates apoptosis studies.

An investigation is conducted to find out the reasons whether i) reduction in cell BrdU incorporation due to treatment with Cannabidiol after transfection with viral proteins as observed earlier and provided in FIGS. 1-6 when cell BrdU incorporation data was not normalized to cell number is due to increased cell apoptosis and whether ii) reduction in relative cell number when cells are transfected with viral proteins and treated with Cannabidiol is due to increased cell apoptosis. In the present study, it has been surprisingly found that Cannabidiol, although it does not exert any significant effect on cells that are transfected with a control plasmid (empty plasmid), exerted unique and significant effects on cells that are transfected with a plasmid expressing the viral Orf8, Orf10 or M proteins. This study unfolds several avenues for use of Cannabidiol for Covid-19.

First, this reflects that the Cannabidiol may be able to differentiate and distinguish between a non-infected and infected cells and act accordingly.

Second, since cells transfected with viral proteins but untreated with Cannabidiol did not show any significant deflection/reduction from a control value, it is possible that interferons are not produced or apoptosis is not induced in such cells. The viral plasmids alone appear to cause only a minor decrease in cell proliferation (or, possibly increases in cell death, or both).

### Apoptosis Studies

Apoptotic cell death is a highly regulated process that is characterized by stereotypical and morphological changes of the cellular architecture including Cell shrinkage, plasma membrane blebbing, cell detachment, externalization of phosphatidylserine, nuclear condensation and ultimately DNA fragmentation (Taylor, R. C. et al, 2008 and Henry, C. M., 2013).

In the early apoptosis, phosphatidylserine concentration rises outside the cell. pSIVA is a marker of early apoptosis that binds to phosphatidylserine, which rises in concentration on the outside of cells when apoptosis begins, and fluoresces after binding. Cells do not yet have to be permeable in order for this interaction to happen.

In late apoptosis, Propidium iodide (PI) is used which binds to DNA, causing fluorescence. PI can only enter cells when they are in a later stage of apoptosis, where the cell and nuclear membranes have become permeable and begun to fragment, which allows PI to enter into cells. This fluorescence is read in a plate reader, which detects pSIVA and PI at different excitation/emission spectra, and so both can be present, but are read separately.

The steps are as follows:

1. HEK293 (human embryonic kidney) cells are chosen for the studies. Cells were seeded in 96-well plates at a density of 10+ cells per well, then grown to 60-70% confluence.

2. Then cells are either transfected with a control plasmid expressing a control vector viz. pCMV-3Tag-3A, or transfected with plasmids expressing ORF8, ORF10, or M-protein. These transfections are done in duplicate.

3. One side is treated with CBD, and one side is treated with ethanol (0.01% v/v final concentration).

3. The pORF8 and pORF10 plasmids express ORF8 or ORF10, each tagged with 3×FLAG tag (so pCMV-3-Tag-3A is essentially a perfect control), while the M-protein is tagged with green fluorescent protein. Thus, pCMV-3Tag-3A represents a control plasmid that is a small, foreign DNA, expressing a small, foreign transcript that is not viral in origin.

4. Twenty-four hours after transfection/treatment, cells were tested for relative rates of apoptosis by adding two markers of apoptosis into the medium viz. pSIVA (for early apoptosis) and propidium iodide (for late apoptosis).

The fluorescence readings gives a relative measure of the proportion of cells in a well that are in either the early stage or late stage of apoptosis at 24 hours.

The experiment is initiated with a fixed number of cells. However, when the experiment is conducted over 24 hrs., due to cell apoptosis, cells get detached and fragmented. Early and late apoptosis markers read adherent cells and hence it is necessary to take a measure of the relative number of cells in a well, when the apoptosis assay is completed.

The density of cells per well is estimated by staining cells after an assay with a cell-stain like crystal violet. Crystal violet is then eluted from cells, and the absorbance per well is measured. The higher the absorbance, the greater the number of cells. The next step is to normalize the measures on apoptosis (i.e. total fluorescence for pSIVA and total fluorescence for PI) to relative cell number, by dividing those fluorescence values by the crystal violet absorbance measures.

FIGS. 8A and 8B provide respectively an early and late apoptosis data of HEK293 (human embryonic kidney) cells transfected with i) control plasmid expressing control vector and ii) plasmid expressing viral protein ORF8; and then treated with Cannabidiol. Cannabidiol treated cells which are transfected with control plasmid do not show any significant change in early as well as late apoptosis but Cannabidiol treated cells which are transfected with plasmid expressing viral protein ORF8 have exhibited significantly greater early apoptosis and late apoptosis.

### ORF8 and Apoptosis

In cells transfected with a control plasmid, CBD did not increase the proportion of cells entering into early or late stages of apoptosis.
However, in cells transfected with ORF8, CBD did significantly enhance apoptosis entry and the proportion of cells found to be in late-stage apoptosis.

The proportion of cells in either early or late stage apoptosis was higher in wells treated with CBD and transfected with ORF8, compared to wells treated with CBD and transfected with control plasmid.
*P<0.05, **P<0.01 indicating significant differences between groups, as marked.

This data is extremely significant for various reasons. First, an early apoptosis only within 24 hrs of transfection indicates that an early cellular defense mechanism has been initiated due to Cannabidiol. Second, if infected host cells undergo apoptosis due to Cannabidiol, then the host machinery is not available for the virus to replicate and mutate. So far no method has been provided to inhibit mutation of virus inside the host cell. Third, since Cannabidiol has not increased early or late apoptosis in cells transfected with only a control plasmid, it is not likely to cause apoptosis of healthy cells. Fourth, ORF8 protein is involved in cellular host-defense evasion and viral pathogenesis. If Cannabidiol can act in the presence of this viral protein in the host's infected cells and cause apoptosis, it can prevent the virus from evading the host's immune system.

This data reflects that if cannabidiol is already present in the human body at the time of virus entry, or is taken at the same time as virus entry, it can even prevent infection due to its early intervention and act prophylactically.

### ORF10 and Apoptosis

FIGS. 12A and 12B provide early apoptosis and late apoptosis data in cells transfected with a control plasmid or viral plasmid expressing ORF10 and treated with Cannabidiol.

Cannabidiol has provided a greater cellular response to ORF10 than to control plasmid, indicating that CBD helps cells to recognize, and respond to SARS-COV-2 genes. Cannabidiol's augmentation of the apoptotic response to ORF10 expression relative to vehicle control is not significant.

### M Protein and Apoptosis

FIGS. 15A and 15B provide early apoptosis and late apoptosis data in cells transfected with a control plasmid or viral plasmid expressing M protein and treated with Cannabidiol.

For the cells expressing M-protein and treated with Cannabidiol, both early and late apoptosis were significantly increased compared to cells expressing M-protein and treated only with vehicle.

CBD did not alter early or late apoptosis significantly in cells expressing only the control plasmid. M-protein expression in Cannabidiol-treated or vehicle-treated cells significantly increased early and late apoptosis relative to these markers in control-transfected cells, respectively. Cannabidiol therefore augmented the pro-apoptotic effect of M-protein.

### Stimulation of Interferons and Interferons Stimulated Antiviral Effectors—ORF8 Protein

The studies involved a third step of Investigation into whether the effects of Cannabidiol are also due to production of interferons in cells transfected with viral proteins. This checks production of interferons and their downstream effectors upon transfection with viral proteins and upon treatment with Cannabidiol.

The study involved estimating interferon lamda-1 levels of cells transfected with a plasmid expressing viral ORF8 and treated with Cannabidiol. The levels are also estimated in cells transfected with a control plasmid expressing control vector and treated with Cannabidiol.

FIG. 9A provides Interferon Lambda 1 mRNA levels produced when cells expressing ORF8 or a control plasmid are treated with Cannabidiol. It also provides comparison of production of Interferon Lambda 1 levels between the cells expressing ORF8, but not treated with CBD and control treated cells not treated with Cannabidiol.

In cells expressing ORF8, but not treated with CBD, Interferon Lambda 1 gene expression was not significantly elevated versus control-treated cells. This highlights the problem that cells often have an inadequate innate anti-viral response to SARS-COV-2.

In cells expressing ORF8, CBD significantly increased expression of Interferon lambda 1 at 24 hours versus treatment with vehicle alone, indicating that CBD augments this anti-viral response to ORF8. However, CBD did not significantly affect INF lambdal expression in cells transfected with a control plasmid, indicating that CBD specifically augments the anti-viral response to a SARS-Cov-2 gene.

Further, levels of Interferon gamma are also studied in cells transfected with control plasmid and plasmid expressing viral proteins. As provided in FIG. 9B, CBD augmented the expression of INF-gamma in both control and ORF8-expressing cells, but had a greater effect on this expression in ORF8 expressing cells.

This finding has immense applications. Cannabidiol is certainly augmenting the innate immune response upon transfection with viral proteins by significantly elevating levels of Interferon lambda 1 in just 24 hours.

Elevation in levels of interferons is essentially an interesting finding. Interferon elevation in the human body as a response to viral entry stimulates interferon stimulated genes also called as interferon stimulated antiviral effectors. If these genes are found in a human body, it is a confirmation of body's augmented immune response and a condition where healthy individuals are better able to fight with the infection and patients are better able to handle Covid-19 infection because the situation would not worsen.

While working on such downstream effector genes, inventors came across such effector genes which are significantly elevated in cells transfected with viral protein particularly ORF8 and treated with Cannabidiol.

As provided in FIG. 10, a highly significant increase in the expression of the OAS1 (Oligoadenylate synthetases 1) gene is found in cells transfected with ORF8 protein and treated with Cannabidiol. Another interferon stimulated gene which is also induced by Cannabidiol plus ORF8 expression is Mx1 (Dynamin-Like GTPase myxovirus resistance protein 1) as provided in FIG. 11. The extent by which CBD augmented OAS1 in ORF8-expressing cells was significantly higher than the extent by which CBD augmented OAS1 expression in cells transfected with control vector. This finding is extremely interesting and exciting and it confirmed the role of Cannabidiol in treating Covid-19 infection.

Interestingly further, very recently Zhou Sirui et al (Zhou Sirui et al, 2021) states as follows:

“ . . . we found that an s.d. increase in OAS1 levels was associated with reduced COVID-19 death or ventilation (odds ratio (OR)=0.54, P=7×10−8), hospitalization (OR=0.61, P=8×10−8) and susceptibility (OR=0.78, P=8×10−6). Measuring OAS1 levels in 504 individuals, we found that higher plasma OAS1 levels in a non-infectious state were associated with reduced COVID-19 susceptibility and severity. Further analyses suggested that a Neanderthal isoform of OAS1 in individuals of European ancestry affords this protection. Thus, evidence from MR and a case-control study support a protective role for OAS1 in COVID-19 adverse outcomes. Available pharmacological agents that increase OAS1 levels could be prioritized for drug development.”

Thus, significant enhancement in levels of OAS1 gene is a confirmation to select Cannabidiol as a therapeutic agent in Drug Development.

More interestingly, the interferon-stimulated genes are not found to be upregulated upon transfection with ORF8 alone, but only with ORF8 and Cannabidiol, although interferon gamma is significantly upregulated by transfection of cells with a plasmid expressing ORF8, even without added Cannabidiol. ORF8 is an accessory protein that has been proposed to interfere with immune responses of the host. The very protein which interferes with the immune response of the host will be unable to exert any effect in the presence of Cannabidiol because in the present case ORF8 is expressed and still, OAS1 has been produced in significant amount.

Particularly, when transfection of cells with plasmid expressing ORF8 protein is carried out without treatment with Cannabidiol, OAS1 gene expression is not significantly elevated compared to levels in vehicle-treated cells transfected with control plasmid. This indicates that an individual exposed to viral protein in absence of Cannabidiol is not able to produce interferons and interferon-induced antiviral effectors such as OAS1 in significant amounts. Also, the fact that Cannabidiol either had a lesser effect, or no effect on control-transfected cells, indicates a high margin of safety.

The data presented in FIG. 11 is also extremely interesting because Cannabidiol in absence of viral protein ORF8 has also produced some amount of OAS1 which means that if Cannabidiol is consumed by healthy individuals not exposed to virus they can also induce interferon transcription and interferon induced antiviral effectors, and become better prepared to respond to a viral threat.

This OAS1 expression can increase more than 10-fold, more than 20-fold and more than 30-fold when viral protein is introduced to make an individual better ready to fight against Covid-19.

### Stimulation of Interferons and Interferon-Stimulated Antiviral Effectors—ORF10 Protein

As provided in FIG. 13, in cells expressing ORF10, CBD significantly increased expression of Interferon gamma which is an indication of augmentation of immunity. Expression of Interferon gamma is also seen in Cannabidiol treated cells transfected with a control plasmid. This expression in the absence of viral protein increases 3-4 folds in presence of viral protein ORF10. Thus, Cannabidiol significantly augments the innate immune response in cells expressing ORF10.

CBD significantly augmented the induction of OAS1 in response to ORF10, compared to cells treated only with vehicle. The OAS1 induction in response to ORF10 plus CBD was lower than the induction in response to CBD plus control plasmid. Nevertheless, CBD did augment this anti-viral response in cells transfected with either plasmid.

### Stimulation of Interferons and Interferons Stimulated Antiviral Effectors—M Protein

As provided in FIGS. 16A and 16B, Cannabidiol induced both INF-lambda 1 and INF-lambda 2/3 in cells expressing M-protein, indicating that Cannabidiol augments the interferon response to this SARS-COV-2 protein and augments the innate immune response. Cannabidiol did not cause an induction of INF-lambda 1, or interferons lambda 2/3 in cells transfected only with control plasmid.

Mx1 is another interferon induced anti-viral effector. As provided in FIG. 17, Cells transfected with M protein and treated with Cannabidiol have higher expression of Mx1 than cells transfected with control vector and treated with Cannabidiol. This indicates a potential that CBD ‘primes’ cells to be ready to respond to a viral threat, upon expression of viral genes. CBD treatment led to enhanced expression of Mx1 in cells expressing M-protein compared to cells overexpressing control plasmid, indicating an enhanced anti-viral response in the presence of this viral gene.

In yet one more interesting study and as provided in FIG. 18, cells transfected with either control plasmid or M protein and treated with cannabidiol have exhibited significant elevations in expression of OAS1 gene relative to vehicle-treated control cells.

Cannabidiol augments interferons and interferon-induced anti-viral effectors even in the absence of viral proteins. This is a strong reason to select Cannabidiol as a prophylactic medicine where CBD may help to prime this aspect of the innate immune response. IFIT1 (interferon-induced protein with tetratricopeptide repeats) is another interferon-induced anti-viral effector. As provided in FIG. 19, Cells transfected with both control plasmid and M protein and treated with cannabidiol have exhibited elevated expression of IFIT1 relative to cells treated with vehicle alone. This augmentation was lower in the cells expressing M-protein than in cells expressing control plasmid, but it was still a significant augmentation, indicating that CBD may help to prime this aspect of the innate immune response.

As reported by Zhou Sirui et al (Zhou Sirui et al, 2021), for treating Covid-19, “Available pharmacological agents that increase OAS1 levels could be prioritized for drug development”. Out of the three viral proteins tested, two have shown a strong reason to select Cannabidiol for Covid-19 therapy.

Surprisingly, the inventors of the present invention came across various facts that reinforce multiple roles of Cannabidiol in prophylaxis and treatment of Covid-19. These roles are summarized below:

### Role in Apoptosis

1. CBD augments early and late apoptosis induction, 24 hours after cells are transfected with viral genes, which suggests that CBD can help cells to fight off an initial infection. Infected host cells apoptose and host machinery is not available for viral replication and mutation. The induction of apoptosis early after viral transcripts appear in a cell is highly protective against infection. Viruses enter cells, and then ‘hijack’ the cellular machinery to begin producing new virus. This is what results in a wide-spread infection, and it is also the time when mutations can be introduced to the viral genome (i.e. during replication of the viral genome), resulting in the emergence of new variants. However, apoptosis causes fragmentation of the cellular organelles, and eventually the cell. If it happens early after infection, it can prevent infected cells from making new virus. This would result in early elimination of virus and infected cells from a person, who would likely not even realize they had been infected.

2. Early apoptosis in cells expressing ORF8 is highly significant as this very protein is proposed to interfere with immune responses of host. ORF8 is unique in that it maybe dispensible in viral replication but it has a unique role of evading immune surveillance of host cells i.e. it has a role in the way virus evades immunity of host cells.

3. CBD does not increase early or late apoptosis in control transfected cells versus vehicle alone, indicating a high degree of safety of CBD and that effects of the combination of CBD and SARS-COV-2 viral proteins are specific.

### Role in Expressing Interferons and Interferons Induced Anti-Viral Effectors

4. Induction of interferons results in an innate, intracellular, anti-viral host defense that does not require immune cells, per se. There are different types of interferons. Type 1 (alpha and beta) tend to slow down proliferation, and also regulate cell survival. Type II (gamma) tends to also regulate both cell survival and proliferation. Type III interferons (i.e. Lambda-type interferons) tend to force cells towards apoptosis, much more so than Type I or II. Although not much change is observed in Type I interferons, some significant increase in Type II and also in Type III interferons are noted as a result of CBD treatment. CBD played dual roles. It expressed interferons in cells transfected with viral proteins but also in cells transfected with a control plasmid and treated with Cannabidiol. Thus it is seen that CBD prepares host for viral threats even in the absence of virus.

5. Some of the interferon induced anti-viral effectors that are expressed during treatment with Cannabidiol include Mx1, IFIT1 and OAS1.

6. IFIT is short for ‘interferon-induced protein with tetratricopeptide repeats’. It binds to RNAs that lack a signature methylation sequence (indicating foreign (and possibly viral) origin) to inhibit their translation—and therefore is an innate cellular mechanism that functions to help stop viral mRNA from being translated into protein. It also “interacts with other cellular proteins to expand their contribution to regulation of the host antiviral response by modulating innate immune signaling and apoptosis.” Inducing IFIT, therefore, should help to slow viral replication. CBD enhanced IFIT1 transcription in control-transfected cells, and in cells expressing M-protein.

7. MX1 (Dynamin-Like GTPase myxovirus resistance protein 1) is an interferon-stimulated gene. This gene can be induced by IFN type I and/or type III (i.e. INF lambda). Mx1 inhibits the transcription of viral RNA. Bizzotto Juan et al (Bizzotto Juan et al, 2020) reports that MX1 levels increase with increasing viral load in SARS COV-2 infection. Mx1 transcription is enhanced by a combination of CBD and ORF8 or CBD and M-protein.

https://pubmed.ncbi.nlm.nih.gov/32989429/8.

8. OAS1 stands for Oligoadenylate synthetases (OAS), which are a family of interferon-stimulated genes that can induce RNA degradation in virus by activating RNaseL.

Zhou et al have reported that higher levels of OAS1 are associated with a Neanderthal SNP in people of European ancestry, and higher levels reduce the risk of COVID-19 death, ventilation, hospitalization or susceptibility.

Out of the three proteins tested, cells transfected with two proteins namely ORF8 and M protein and treated with Cannabidiol exhibited significantly increased expression of the OAS1 gene. This makes Cannabidiol a confirmed candidate for treating Covid-19.

OAS1 when produced will activate endoribonuclease L (RNAse L), which degrades all cellular RNA—including both viral and cellular. This results in apoptosis, which is evident in the present case. This effect is much bigger and much more significant than just an anti-viral or replication inhibition effect that allows for cell survival. OAS1 transcript levels were significantly enhanced by treatment with CBD in cells expressing control plasmid, or ORF8, ORF10, or M-protein, versus vehicle control treatment. This would be expected to significantly enhance apoptosis in response to viral presence, or to prime cells to be prepared for a viral infection, allowing a more rapid anti-viral, pro-apoptotic response to viral infection. Augmenting induction of OAS1 gene is associated with a dramatic protection against SARS-COV-2 (and people with higher expression are less likely to get sick).

Thus, to conclude, Cannabidiol has multiple pathways through which it enhances immune response of the host cells. It prepares host cell for viral threat and can act as a prophylactic medicine. The minor increase in OAS1 expression and INF-gamma in control-transfected cells treated with CBD indicates a potential that CBD ‘primes’ cells to be ready to respond to a viral threat, without actually increasing apoptosis. On the other hand, remarkable increase in interferons and interferon-induced effector genes have been found to enhance immune response of the cell when cells transfected with viral proteins are treated with Cannabidiol. Cannabidiol causes early and late apoptosis in cells transfected with ORF8 and M protein. Whether apoptosis is due to Cannabidiol alone or through increased levels of Type III interferons (i.e. Lambda-type interferons), which tend to force cells towards apoptosis, it makes infected host cells not available to the virus to replicate and mutate.

### Cannabidiol

Cannabidiol can also improve outcomes of existing immunization strategies for COVID-19 including but not restricted to by reducing the chances of transmission of viral particles following vaccination and before a full immune response in the individual is mounted, while also preventing the expansion of the viral gene pool through prevention of mutation.

Indeed, even after immunity has been acquired from vaccination, individuals who contract SARS-COV-2 can still generate novel variants when mutation occurs during viral replication, since viral replication occurs during the time between cell infection, and activation of an effective and full humoral acquired (also called adaptive) immune response. This activation can take hours to days, and therefore even vaccinated people can still spread the virus, and produce mutants, during this interval. By augmenting apoptosis in cells exposed to viral genes, Cannabidiol can prevent viral replication and therefore the formation of novel SARS-COV-2 variants.

Cannabidiol can also be candidate for including but not restricted to prophylaxis for travelers, essential workers and other high risk individuals to potentially control the spread of the virus within the host as well as transmission to others. Further the potential to prevent mutations becomes significant, especially for travelers who may be susceptible to introducing non indigenous strains into new geographies which may increase variants.

Cannabidiol also has regulatory approval for pediatric use in patients as young as 1 year of age for rare forms of epilepsy. Therefore, its potential for use in children who may be asymptomatic carriers and/or reservoirs of Sars-COV-2 and other viruses, cannot be undermined, for prophylaxis, to reduce chances of community spread and increased variants and mutations. Cannabidiol can potentially be also administered to new born babies as a mono-treatment with Cannabidiol or as a prophylactic or as an adjunct therapy for Sars-COV-2 and other viruses.

A suitable dose/therapeutically effective amount of Cannabidiol (CBD) is from 0.00001 mg/kg of body weight to 4000 mg/kg of body weight. The suitable dose/therapeutically effective amount of Cannabidiol can also be 0.00001 to 1000 mg/kg of body weight or 0.00001 to 500 mg/kg of body weight. The preferred dose/preferred therapeutically effective amount of Cannabidiol can be 0.00001 to 100 mg/kg of body weight or from 0.00001 to 10 mg/kg of body weight.

The dose will depend on the nature and status of human or animal patient health. It will also depend on age and comorbidities if any. Further, dose will depend on type of composition for example, whether oral or parenteral or topical.

Following pharmaceutical formulations/compositions are described for better understanding of the invention and they do not limit scope of the invention in any way.

### Pharmaceutical Compositions:

Suitable oral dosage forms include but are not restricted to tablets—sublingual, buccal, effervescent, chewable; troches, lozenges, dispersible powders or granules and dragees; capsules, solutions, suspensions, syrups, lozenges, medicated gums, buccal gels or patches. Tablets can be made using compression or molding techniques well known in the art. The other dosage forms can also be prepared by 3Dimensional (3D) or 4D printing and also by Carbon graphene loaded nano-particles and micro-particles. Gelatin or non-gelatin capsules can be formulated as hard or soft capsule shells, which can encapsulate liquid, solid, and semisolid fill materials, using techniques well known in the art.

Following examples provide various Pharmaceutical compositions of the Cannabidiol (CBD)

The Oral Spray formulation encompasses the Cannabidiol (CBD); each at concentration of 0.00001 mg to 200 mg/ml and have excipients such as diluents viz. Mannitol ranging from 10-15 mg/ml; Sweeteners such as sucralose from 5-10 mg/ml, Flavours as Raspberry, Strawberry from 5-10 mg/ml and tonicity and taste modulators such as sodium chloride and propylene glycol from 0.1-0.5 mg/ml with purified water as the base solvent or carrier. The specific gravity of the formulation can be between 1.01 to 1.5 g/ml

Additionally, the said Oral Spray may encompass surfactant-solubilizers and gelling agents such as Pluronic F127 or Poloxamer 407 in the concentration ranging from 1-200 mg/ml. This formulation is liquid at temperatures less than 10 degree Celsius and starts gelling at temperature range above 30 degree Celsius. It is a sterile, nonpyrogenic solution. The pH range if reconstituted should be 5-9 preferably 6.5-7.5. It can be administered using appropriate spray containers with specialised spray nozzle to facilitate spray below the tongue viz. sublingually or into the buccal or also the nasal cavity. The Spray can also be in the form of a micronized or nanosized suspension. The Nasal Spray formulation would be devoid of the sweeteners and flavours. It gels at body temperature thereby facilitating longer dwell time possibly enhancing the drug penetration through the mucosal lining. This drug delivery mode bypasses the harsh acidic conditions of the stomach and also the hepatic breakdown thereby possibly increasing bio-availability. The specific gravity of the formulation can be between 1.01 to 1.7 g/ml

The Injection formulation contains the Cannabidiol (CBD); at concentration of 0.00001 mg to 200 mg/ml and solubilizers such as Ethyl alcohol 20%/ml and Propylene glycol 40%/ml and Water for injection ˜40%/ml. The solution should be isotonic and tonicity adjusting salts such as sodium chloride can be used. The pH range of 5-9 can be adjusted with suitable bufferants should be 6-8 preferably 6.5-7.5. It is a sterile, nonpyrogenic solution. The said Injection formulation can be in the form of a solution or micronized or nanosized dispersion. The said formulation can also be administered via inhalation with or without the aid of a medical device, metered or unmetered, and/or via nebulization for nasal administration for drug delivery to the lungs—viz. Pulmonary. The said formulation can also be administered via the buccal route as buccal drops or as buccal spray using appropriate medical device. The said formulation can be administered via the sublingual route as sublingual drops or as sublingual spray using appropriate medical device. Another variant of the sterile injectable formulation can also be a lyophilized injection. This injection may also contain sodium citrate dihydrate and citric acid anhydrous; and finally, be as a white to yellow lyophilized powder or plug. The solution should be prepared only with 1 to 2 mL of preservative-free Sterile Sodium Chloride Injection, 0.9 percent or preservative-free Sterile Water for Injection. The reconstituted solution is clear, slightly yellow and essentially free from visible particles. The specific gravity of the formulation can be between 1.01 to 1.7 g/ml. The particle size of the liquid droplets can range from 5 micron to 500 micron.

The Inhalation or Pulmonary Capsule has the Cannabidiol (CBD) concentration of 0.00001 mg to 50 mg/capsule and has excipients such as Magnesium stearate [Inhalation grade] or Lactose [Inhalation grade]. The core weight of the formulation can range from 25-500 mg/capsule.

The Aerosol or Pulmonary delivery system has the Cannabidiol (CBD) concentration of 0.00001 mg to 100 mg/actuation and has excipients such as propellent gases, propylene glycol, water, surfactants, anti-foam emulsion and anti-freeze excipients. The particle size of the liquid droplets can range from 5 micron to 500 micron.

Sublingual Tablets have the Cannabidiol (CBD); at concentration of 0.00001 mg to 50 mg/tablet and have excipients such as diluents viz. Lactose monohydrate or Mannitol ranging from 10-30 mg/tablet;

Disintegrants such as Starch or Crospovidone from 10-15 mg/tablet; fillers such as Microcrystalline cellulose from 5-10 mg/tablet and lubricants such as Magnesium stearate from 0.5-1 mg/tablet. It may additionally contain or 5-10 mg/tablet of taste modulating or masking agents such as sodium chloride or buffers such as potassium dihydrogen phosphate. The core weight of the formulation can range from 50-80 mg/tablet.

The Orally Dispersible Tablets (ODT) have the Cannabidiol (CBD) at concentration of 0.00001 mg to 100 mg/tablet and have excipients such as diluents viz. Lactose monohydrate or Mannitol ranging from 10-15 mg/tablet; Disintegrants such as Starch or Crospovidone from 10-15 mg/tablet; fillers such as Microcrystalline cellulose from 5-10 mg/tablet and lubricants such as Magnesium stearate from 0.5-1 mg/tablet. The core weight of the formulation can range from 50-80 mg/tablet.

The Buccal Tablets have the Cannabidiol (CBD) at concentration of 0.00001 mg to 100 mg/tablet and have excipients such as polymers viz. polymers of acrylic acid and C10-C30 alkyl acrylate crosslinked with allyl pentaerythritol exemplified by Carbopol 934 ranging from 10-15 mg/tablet and or Hydroxy Propyl Methyl Cellulose (HPMC) K4M from 35-40 mg/tablet; Fillers such as Mannitol (directly compressible) from 10-15 mg/tablet; and lubricants such as Magnesium stearate from 0.5-1 mg/tablet. The core weight of the formulation can range from 50-80 mg/tablet.

The Delayed Release Tablets have the Cannabidiol (CBD) at concentration of 0.00001 mg to 200 mg/tablet and have excipients such as Mannitol, Microcrystalline cellulose (MCC PH 102), trisodium phosphate, Hydroxy Propyl Methyl Cellulose (HPMC 5 cps), Hydroxy Propyl Methyl Cellulose (HPMC 15 cps) and Crospovidone, Colloidal silicon dioxide, Magnesium stearate as the tablet core coated with a Seal Coating composition encompassing Ethyl cellulose using an appropriate solvent system viz. aqueous, non-aqueous; preferably non-aqueous (Iso-propyl alcohol and Dichloromethane) to a 4-5% weight gain on the tablet cores finally coated with an aqueous gastro-resistant coating composition viz. Eudragit L100-55, Triethyl citrate, opacifier and colorant to a total weight gain of 26-30% of the tablet cores. The core weight of the formulation can range from 50-1200 mg/tablet.

The Extended Release Tablets have the Cannabidiol (CBD) at concentration of 0.00001 mg to 200 mg/tablet and have excipients such as fillers viz. Microcrystalline cellulose (MCC PH 101); polymers viz Hydroxy Propyl Methyl Cellulose (HPMC K100M) and Hydroxy Propyl Methyl Cellulose (HPMC K15M); binders viz. Povidone (PVP K29/32) and Lubricants viz. Magnesium stearate as the tablet core coated with a Film Coating composition using an appropriate solvent system viz. aqueous or non-aqueous; preferably non-aqueous (Iso-propyl alcohol and Dichloromethane) to a 2-3% weight gain on the tablet core. The core weight of the formulation can range from 50-1200 mg/tablet.

The Effervescent tablets have the Cannabidiol (CBD) at concentration of 0.00001 mg to 200 mg/tablet and have excipients such as citric acid, sodium bicarbonate, potassium citrate, mannitol, aspartame, strawberry flavour, bufferants, sodium benzoate and polyethylene glycol 6000. The core weight of the formulation can range from 50-2000 mg/tablet.

The Osmotic-controlled Release Oral delivery System (OROS) Tablets have the Cannabidiol (CBD) at concentration of 0.00001 mg to 200 mg/tablet and have excipients such as sorbitan monolaurate and Sodium chloride, microcrystalline cellulose (MCC PH 102), polymers viz Hydroxy Propyl Methyl Cellulose (HPMC K100M) and Hydroxy Propyl Methyl Cellulose (HPMC K15M), Colloidal silicon dioxide and Magnesium stearate as the tablet core; a Film coat to the tablet cores to a weight gain of 2.5 to 3.0% w/w to the tablet core using a non-aqueous medium and a Functional Coat the tablet with Cellulose acetate non-aqueous dispersion in Iso-propyl alcohol to a weight gain of 25-30% w/w of the tablet core finally Laser drilled the tablets with an orifice of 150-250 micron. The core weight of the formulation can range from 50-1000 mg/tablet.

The Capsules have the Cannabidiol (CBD) at concentration of 0.00001 mg to 200 mg/capsule and have excipients such a microcrystalline cellulose (MCC PH 105), Colloidal silicon dioxide and Magnesium stearate as the core; encompassed in a hard gelatin capsule. The core weight of the formulation can range from 30-2055 mg/capsule.

The Compressed lozenges or Chews or Lollipop have the Cannabidiol (CBD) at concentration of 0.00001 mg to 200 mg/unit and have excipients such as ethoxylated hydrogenated castor oil Polyoxyl 35 Castor oil (Cremophore EL/Kolliphor EL), Dextrate, Polyethylene glycol 6000, microcrystalline cellulose (MCC 102), povidone (PVP K29/32) and FD&C Yellow No. 6 and Magnesium stearate as the core. The core weight of the formulation can range from 100-3000 mg/unit

The Soft Gel Capsules have the Cannabidiol (CBD) at concentration of 0.00001 mg to 200 mg/capsule and have excipients such as propylene glycol, Poly Ethylene Glycol-400, Polyvinyl pyrrolidone K29/32, Butylated hydroxy toluene and ethanol-water blend as the core material filled into opaque soft gelatin capsules. The core weight of the formulation can range from 100-800 mg/capsule.

The Quick dissolving film—Oral and or Sublingual, have the Cannabidiol (CBD) at concentration of 0.00001 mg to 200 mg/unit and have excipients such as pullulan, sorbitol, polysorbate 80, sucralose, Monoammonium glycyrrhizinate and peppermint flavour. The core weight of the formulation can range from 50-800 mg/unit

The Oro-Buccal muco-adhesive film—Oral or Sublingual, have the Cannabidiol (CBD) at concentration of 0.00001 mg to 200 mg/unit and have excipients such as Hydroxypropyl cellulose, Hydroxyethyl cellulose and Sodium carboxymethyl cellulose, Polyoxyl 35 Castor Oil (Cremophore EL/Kolliphor EL), Sodium benzoate, Parahydroxybenzoate methyl, Parahydroxybenzoate propyl, Sodium citrate and Sodium saccharine. The core weight of the formulation can range from 50-80 mg/unit.

The Oral Emulsion has the Cannabidiol (CBD) at concentration of 0.00001 mg to 200 mg/g and have excipients such as Polyoxyl 35 Castor Oil (Cremophore EL/Kolliphor EL), Saccharin Sodium, caramel, colorant, peppermint oil, corn oil, sucrose and water. The specific gravity of the formulation can be between 0.5-1.5 g/ml

The Vaginal gel has the Cannabidiol (CBD) at concentration of 0.00001 mg to 200 mg/g and has excipients such as Polyoxyl 35 Castor Oil (Cremophore EL/Kolliphor EL), ascorbic acid, Glycerin or Propylene glycol, Hydroxypropyl Methylcellulose (HPMC E50), Trisodium Citrate dihydrate and water. The specific gravity of the formulation can be between 1.01-1.8 g/ml.

The Eye drop formulation has the Cannabidiol (CBD) at concentration of 0.00001 mg to 200 mg/ml and has excipients such as Polysorbate 20/80, Benzalkonium chloride, disodium EDTATE, Sodium Carboxymethyl Cellulose (Na CMC), Citric acid monohydrate, sodium hydroxide, hydrochloric acid and water. The final solution is sterile. The specific gravity of the formulation can be between 1.01-1.8 g/ml.

The Suppository formulation has the Cannabidiol (CBD) at concentration of 0.00001 mg to 200 mg/g and has excipients such as hard fat, surfactants, and the following inactive ingredients: butylated hydroxy anisole, butylated hydroxytoluene, edetic acid, glycerin, polyethylene glycol 3350, polyethylene glycol 8000, purified water and sodium chloride. The core weight of the formulation can range from 200-3000 mg/unit

### EXAMPLES

### Example 1: Process for Measuring Cell Proliferation Rates

The bromodeoxyuridine incorporation rate was measured by incorporating and quantifying bromodeoxyuridine (BrdU) into DNA of actively proliferating cells. The absorbance values are measured by ELISA assay with a BioTek Synergy H1 Hybrid Multi-Mode Microplate reader assay at 370 nm (reference wavelength: approx. 492 nm). FIGS. 1-5 provide results of the tests performed. Note, however, that cell proliferation level can only be inferred once the data are normalized to cell number. FIG. 6 combines data from all figures for ready comparison. The absorbance is expressed as % untreated control. In FIG. 7, data are normalized to relative cell number.

### Example 2—Crystal Violet Staining

Relative cell numbers were quantified using the crystal violet staining, as previously described by Duncan, R. E., et al [Duncan, R. E., et al, 2004]. Briefly, cells seeded in 96-well plates were gently washed with 1× phosphate buffered saline (PBS) after BrdU incorporation assay or Apoptosis assay, fixed with 10% methanol 10% acetic acid and stained with crystal violet. Absorbance of the samples was measured in a plate reader at 595 nm.

### Example 3: Apoptosis Assay

Detection of apoptotic cells was performed using the Apoptosis Assay Kit (Abcam, ab 129817), according to the manufacturer's instructions. Briefly, 24 h after transfection, cultured cells seeded in 96-well plates were labelled with Polarity Sensitive Indicator of Viability & Apoptosis (pSIVA, which detects early/ongoing apoptosis) and with Propidium Iodide (PI, which detects late apoptosis). Fluorescence of the samples was measured in a plate reader at 469/525 nm (for the detection of pSIVA) and 531/647 nm (for the detection of PI).

### Example 4: Methods for Measuring Levels of Interferons and Effectors Gene Expression

Reverse transcriptase—realtime quantitative polymerase chain reaction (qPCR) analysis was conducted as we have previously described. Cells were grown in 24 well plates, and transfected with either pCMV-3Tag-3A as a control vector, or plasmids expressing ORF8, ORF10, or M-protein, and 6 hours later they were treated with 1 μM CBD or vehicle control (0.01% ethanol) for 24 hours. Briefly, total RNA was isolated from cells using TRizol® Reagent as described by the manufacturer (Invitrogen, Waltham, MA). Quantification of RNA samples was performed using a Nanodrop 2000 Spectrophotometer (Thermo Fisher, Waltham, MA), and 2 μg of RNA was used to synthesize cDNA via oligo(dT) priming using the SuperScript II Reverse Transcriptase, according to the manufacturer's protocol (Invitrogen, Waltham, MA). For the real-time PCR assays, cDNA was diluted 1:4 and 1 μ1 was added to a master mix with 9 μ1 of PerfeCTa SYBR® Green supermix (Quanta Bio, Beverly, MA), 0.5 μ1 forward and reverse primers (25 UM each) of the targeted gene (please see list below), and 3 μ1 of ddH20. The cycling conditions for all genes were as follows: 1 cycle of 95° C. for 2 min, followed by 49 cycles of 95° C. for 10 s, then 60° C. for 20 s. Relative expression of the targeted gene was calculated using the delta-delta-Ct method with the Ct values normalized to Glyceraldehyde 3-phosphate dehydrogenase (Gapdh).

Primer sequence:
IFN-gamma-Forward
(SEQ ID No: 1)
5′-TGGCTTTTCAGCTCTGCATC-3′
IFN-gamma-Reverse
(SEQ ID No: 2)
5′-CCGCTACATCTGAATGACCTG-3′
IFN-epsilon 1-Forward
(SEQ ID No: 3)
5′-GAGGCCCCCAAAAAGGAGTC-3′
IFN-epsilon1-Reverse
(SEQ ID No: 4)
5′-AGGTTCCCATCGGCCACATA-3′
IFN epsilon 2-3-forward
(SEQ ID No: 5)
5′-CTGCCACATAGCCCAGTTCA-3′
IFN epsilon 2-3-reverse
(SEQ ID No: 6)
5′-AGAAGCGACTCTTCTAAGGCATCTT-3′
Mx 1-forward
(SEQ ID No: 7)
5′-TCT GAG GAG AGC CAG ACG AT-3′
Mx1-reverse
(SEQ ID No: 8)
5′-ACT CTG GTC CCC AAT GAC AG-3′
OAS1-forward
(SEQ ID No: 9)
5′-GGA TGC CTG GGA GAG AAT CG-3′
OAS1-reverse
(SEQ ID No: 10)
5′-TCG CCT GCT CTT CGA AAC TG-3′
IFITI QI Forward
(SEQ ID No: 11)
5′-GGAATACACAACCTACTAGCC-3′
IFITI QI Reverse
(SEQ ID No: 12)
5′-CCAGGTCACCAGACTCCTCA-3
hGapdh-forward
(SEQ ID No: 13)
5′-AGAAGGCTGGGGCTCATTTG-3′
hGapdh-reverse
(SEQ ID No: 14)
5′-AGGGGCCATCCACAGTCTTC-3′

Example 5—CANNABIDIOL FILM COATED TABLETS

| A | TABLET CORE | |
|---|---|---|
| 1 | Cannabidiol | 0.1 mg to 100 mg or |
| | | 100 mg to 200 mg |
| 2 | Microcrystalline cellulose | 40% of the total core |
| | (MCC PH105) | weight |
| 3 | CELLULOSE METHYLHYDROXY- | 2% of the total core |
| | PROPYL 5CPS | weight |
| 4 | COLLOIDAL SILICON DIOXIDE | 2% of the total core |
| | | weight |
| 5 | POLYVINYL PYROLLIDONE | 2% of the total core |
| | (PVP K29/32) | weight |
| 6 | MAGNESIUM STEARATE | 0.5% of the total core |
| | | weight |
| B | FILM-COATING | |
| | Consists of Polyvinyl alcohol, poly- | 2.0-2.5% of the total |
| | ethylene glycol, Talc, Opacifier, | core weight |
| | lecithin reconstituted to 10% w/w | |
| | dispersion in water-Iso-propyl alcohol | |
| | blend *or Iso-propyl alcohol* | |
| | *= Evaporates during tablet coating and | |
| | is not present substantially in the final | |
| | product—The film coated tablet. | |
| C | PROCESS: | |

Co-sift Cannabidiol and MCC PH 105, Cellulose methyl
hydroxypropyl and polyvinyl-pyrollidone through
ASTM # 40 mesh twice. Label it as Mix A.
Sift individually the colloidal silicon dioxide and the
magnesium stearate through ASTM # 40 and
collect in separate polybags.
Transfer the Mix A to a V-blender of appropriate size
allowing 60% of its occupancy. Blend at 15 RPM for
10 minutes. Label it as Mix B.
Add the pre-sifted colloidal silicon dioxide the Mix B in
the blender and continue to blend at 10 RPM for 5
minutes. Label it as Mix C.
Add the pre-sifted magnesium stearate to the Mix C in
the blender and continue to blend at 10 RPM for 2 minutes.
Unload the final blend into a double LDPE polybag lined
with a black polybag on the outermost side. Displace the
air inside each bag and tie each one with a nylon tag. Keep
5 dessicant pillow pouches (each with 100 gm capacity) in
the second outer bag before tying it up with a nylon tie.
Finally, put the double polybag with the Mix into a black
polybag and secure with a nylon tie. Label the final bag as
“Lubricated Blend ready for compression”. Use appropriate
compression tooling to compress the Lubricated Blend into
biconvex tablets of appropriate hardness so that the percent
friability is less than 0.5% w/w and the disintegration time
(DT) is not more than 15 minutes.
Further coat the tablets in an appropriate tablet coater/
coating machine with the hydro-alcoholic or alcoholic
tablet coating dispersion of appropriate sprayable
consistency to achieve a weight gain of 2.0-2.5% w/w on the
tablet core.
Fill the film-coated tablets into appropriate well-filled, opaque
white/coloured containers (of appropriate material) so
that there is minimum head space along with appropriate
protectants against moisture and oxygen.

Example 6: CANNABIDIOL CAPSULES

| A | CORE INGREDIENTS | |
|---|---|---|
| 1 | Cannabidiol | 0.1 mg to 100 mg or |
| | | 100 mg to 200 mg |
| 2 | Microcrystalline cellulose | 40% of the total |
| | (MCC PH105) | capsule core weight |
| 3 | CELLULOSE | 2% of the total |
| | METHYLHYDROXYPROPYL 5CPS | capsule core weight |
| 4 | COLLOIDAL SILICON DIOXIDE | 2% of the total |
| | | capsule core weight |
| 5 | POLYVINYL PYROLLIDONE | 2% of the total |
| | (PVP K29/32) | capsule core weight |
| 6 | MAGNESIUM STEARATE | 0.5% of the total |
| | | capsule core weight |
| B | ENCAPSULATION | |
| | Consisting of opaque, coloured, | |
| | Hydroxy-propyl methyl cellulose | |
| | (HPMC) of appropriate size | |
| | viz. 00el to 5 to encompass or | |
| | encapsulate the ingredients. | |

| C | PROCESS: |
|---|---|
| | Co-sift Cannabidiol and MCC PH 105, cellulose methyl |
| | hydroxypropyl and polyvinyl-pyrollidone through |
| | ASTM # 40 mesh twice. Label it as Mix A. |
| | Sift individually the colloidal silicon dioxide and the |
| | magnesium stearate through ASTM # 40 and collect in |
| | separate polybags. |
| | Transfer the Mix A to a V-blender of appropriate size |
| | allowing 60% of its occupancy. Blend at 15 RPM for |
| | 10 minutes. Label it as Mix B. |
| | Add the pre-sifted colloidal silicon dioxide the Mix B in |
| | the blender and continue to blend at 10 RPM for 5 |
| | minutes. Label it as Mix C. |
| | Add the pre-sifted magnesium stearate to the Mix C in |
| | the blender and continue to blend at 10 RPM for 2 |
| | minutes. |
| | Unload the final blend into a double LDPE polybag lined |
| | with a black polybag on the outermost side. Displace the |
| | air inside each bag and tie each one with a nylon tag. Keep |
| | 5 dessicant pillow pouches (each with 100 gm capacity) in |
| | the second outer bag before tying it up with a nylon tie. |
| | Finally, put the double polybag with the Mix into a black |
| | polybag and secure with a nylon tie. Label the final bag as |
| | “Lubricated Blend ready for Capsule filling”. Use |
| | appropriate tooling and the Capsule filling machine to fill |
| | the Lubricated Blend into capsules of appropriate size |
| | such that the disintegration time (DT) is not more than |
| | 10 minutes. Label them as Finished Capsules. |
| | Fill the finished capsules into appropriate well-filled, |
| | opaque white/coloured containers (of appropriate material) |
| | so that there is minimum head space along with |
| | appropriate protectants against moisture and oxygen. |

Example 7
CANNABIDIOL INJECTION or
CANNABIDIOL nasal drops or
CANNABIDIOL nasal spray or
CANNABIDIOL buccal drops or
CANNABIDIOL buccal spray or
CANNABIDIOL sublingual drops or
CANNABIDIOL sublingual spray

| | | |
|---|---|---|
| 1 | Cannabidiol | 0.5-100 mg/ml |
| 2 | Propylene glycol | 30% |
| 3 | Ethyl alcohol | 20% |
| 4 | Sodium benzoate/benzoic acid | 5% |
| 5 | Benzyl alcohol | 1.50% |
| 6 | Water for injection | ~43% |

It is a sterile, nonpyrogenic solution. The pH range if
reconstituted should be 5-9 preferably 6.5-7.5
Dissolve the Cannabidiol in ethanol under continuous
stirring in a closed vessel.
Label it as Mix A.
Add the sodium benzoate/benzoic acid and benzyl
alcohol to propylene glycol under continuous stirring
in a larger vessel. Slowly add water to it under stirring.
Label it as Mix B.
Add the Mix B to mix A under continuous stirring.
Continue stirring till a clear solution is formed. Filter
the final clear solution through a 0.2-micron filter
All activity is to be executed in a parenteral facility using
aseptic process only.
Using aseptic filling fill and seal the sterile solution into
ampoules of 1 ml capacity under nitrogen purging and
under subdued light or under a sodium vapour lamp.
The said formulation can be administered via the nasal
route as nasal drops or as nasal spray using appropriate
medical device.
The said formulation can be administered via inhalation
with or without the aid of a medical device, metered or
unmetered, and/or via nebulization.
The said formulation can be administered via the buccal
route as buccal drops or as buccal spray using
appropriate medical device.
The said formulation can be administered via the
sublingual route as sublingual drops or as sublingual
spray using appropriate medical device.

Example 8
CANNABIDIOL INJECTION or
CANNABIDIOL nasal drops or
CANNABIDIOL nasal spray or
CANNABIDIOL buccal drops or
CANNABIDIOL buccal spray or
CANNABIDIOL sublingual drops or
CANNABIDIOL sublingual spray

| | | |
|---|---|---|
| 1 | Cannabidiol | 0.5-100 mg/ml (active) |
| 2 | Ethyl alcohol | 20% of the active |
| 3 | Propylene glycol | 40% of the active |
| 4 | Water for injection | ~40% |
| | It is a sterile, nonpyrogenic | |
| | solution with pH range 4.0-7.0. | |
| | The pH range if reconstituted | |
| | should be 5-9 preferably 6.5-7.5 | |

Dissolve the Cannabidiol in ethanol under continuous
stirring in a small vessel.
Label it as Mix A.
Add the propylene glycol to mix A under continuous
stirring in a larger vessel.
Slowly add water to it under stirring.
Continue stirring till a clear solution is formed. Filter
the final clear solution through a 0.2-micron filter
to yield sterile solution.
All activity is to be executed in a parenteral facility
using aseptic process only.
Using aseptic filling fill and seal the sterile solution
into ampoules of 1 ml capacity under nitrogen purging
and under subdued light or under a sodium vapour
lamp.
The said formulation can be administered via the nasal
route as nasal drops or as nasal spray using
appropriate medical device.
The said formulation can be administered via inhalation
with or without the aid of a medical device, metered
or unmetered, and/or via nebulization.
The said formulation can be administered via the buccal
route as buccal drops or as buccal spray using
appropriate medical device.
The said formulation can be administered via the
sublingual route as sublingual drops or as sublingual
spray using appropriate medical device.

Example 9—CANNABIDIOL ear drops

| | | |
|---|---|---|
| 1 | Cannabidiol | 0.1-100 mg/ml |
| 2 | Iso-propyl alcohol | 95% |
| 3 | Glycerin | 5% |

Dissolve the Cannabidiol in iso-propyl alcohol under
continuous stirring in a closed vessel. Add the glycerin
under continuous stirring. Continue stirring till a clear
solution is formed. Filter the final clear solution
through a 0.2-micron filter to yield a sterile solution.
All activity is to be executed in a parenteral facility
using aseptic process only. Using aseptic filling fill and
seal the sterile solution into dark amber colored glass
vials or appropriate opaque-to-light containers of
suitable material of 10 ml capacity under nitrogen
purging and under subdued light or under the light of a
sodium vapour lamp.
The said formulation can be administered via the
auricular or otic route as ear drops or can also be
alternatively be administered as an intra-auricular spray
using an appropriate medical device.

Example 10—CANNABIDIOL ear drops

| | | |
|---|---|---|
| 1 | Cannabidiol | 0.1-100 mg/ml |
| 2 | Propylene glycol | 95% |

Dissolve the Cannabidiol in propylene glycol under
continuous stirring in a closed vessel.
Continue stirring till a clear solution is formed. Filter
the final clear solution through a 0.2-micron filter
to yield a sterile solution.
All activity is to be executed in a parenteral facility
using aseptic process only.
Using aseptic filling fill and seal the sterile solution
into dark amber colored glass vials or appropriate
opaque-to-light containers of suitable material of 10
ml capacity under nitrogen purging and under subdued
light or under the light of a sodium vapour lamp.
The said formulation can be administered via the
auricular or otic route as ear drops or can also
alternatively be administered as an intra-auricular
spray using an appropriate medical device.

Example 11—CANNABIDIOL ear drops

| | | |
|---|---|---|
| 1 | Cannabidiol | 0.1-100 mg/ml |
| 2 | Industrial Methylated Spirits | 75% |
| | 95% (IMS) | |
| 3 | Glycerin | 5% |
| 4 | Polysorbate 80 | 2.5% |
| 5 | Sodium Hydroxide (for pH- | Quantity sufficient |
| | adjustment) | |
| 6 | Hydrochloric Acid (for pH- | Quantity sufficient |
| | adjustment) | |
| 7 | Purified Water | Quantity sufficient |
| | | for 100% |

Dissolve the Cannabidiol, glycerin and polysorbate 80
in the IMS under continuous stirring in a closed vessel.
Add 90% the purified water to the solution under
stirring. Adjust the pH of the solution with 1N sodium
hydroxide solution and 1N hydrochloric acid to a pH
range between 6-7 under stirring. Add the remaining
water to the solution under continuous stirring.
Continue stirring till a clear solution is formed. Filter
the final clear solution through a 0.2-micron filter to
yield a sterile solution. All activity is to be executed in
a parenteral facility using aseptic process only. Using
aseptic filling fill and seal the sterile solution into dark
amber colored glass vials or appropriate opaque-to-
light containers of suitable material of 10 ml capacity
under nitrogen purging and under subdued light or
under the light of a sodium vapour lamp.
The said formulation can be administered via the
auricular or otic route as ear drops stored in a dark,
amber colored, opaque-to-light container or can also
be alternatively be administered as an intra-auricular
spray using an appropriate medical device.

Example 12—CANNABIDIOL ear drops

| | | |
|---|---|---|
| 1 | Cannabidiol | 0.1-100 mg/ml |
| 2 | Polyethylene glycol | 75% |
| 3 | Sodium Hydroxide (for pH-adjustment) | Quantity sufficient |
| 4 | Hydrochloric Acid (for pH-adjustment) | Quantity sufficient |
| 5 | Purified Water | Quantity sufficient |
| | | for 100% |

Dissolve the Cannabidiol in the polyethylene glycol
under continuous stirring in a closed vessel. Add 90%
the purified water to the solution under stirring. Adjust
the pH of the solution with 1N sodium hydroxide
solution and 1N hydrochloric acid to a pH range
between 6-7 under stirring. Add the remaining
water to the solution under continuous stirring.
Continue stirring till a clear solution is formed. Filter
the final clear solution through a 0.2-micron filter
to yield a sterile solution. All activity is to be executed
in a parenteral facility using aseptic process only.
Using aseptic filling fill and seal the sterile solution into
dark amber colored glass vials or appropriate opaque-to-
light containers of suitable material of 10 ml capacity
under nitrogen purging and under subdued light or under
the light of a sodium vapour lamp.
The said formulation can be administered via the auricular
or otic route as ear drops or can also alternatively be
administered as an intra-auricular spray using an
appropriate medical device.

### REFERENCES

Lu R, Zhao X, Li J, et al. Genomic characterisation and epidemiology of 2019 novel coronavirus: implications for virus origins and receptor binding. Lancet (London, England). 2020 February; 395(10224):565-574. DOI: 10.1016/s0140-6736(20)30251-8.
Zhou, P. et al, A pneumonia outbreak associated with a new coronavirus of probable bat origin, Nature. 2020 March; 579(7798):270-273. doi: 10.1038/s41586-020-2012-7. Epub 2020 Feb. 3.
Xu J, Zhao S, Teng T, et al. Systematic Comparison of Two Animal-to-Human Transmitted Human Coronaviruses: SARS-COV-2 and SARS-COV. Viruses. 2020; 12(2):244. Published 2020 Feb. 22. doi:10.3390/v12020244
Shi C S, Qi H Y, Boularan C, Huang N N, Abu-Asab M, Shelhamer J H, et al. SARS-coronavirus open reading frame-9b suppresses innate immunity by targeting mitochondria and the MAVS/TRAF3/TRAF6 signalosome. Journal of immunology. 2014; 193(6):3080-9.
Jin-Yan Li et al-Jin-Yan Li, Ce-Heng Liao, Qiong Wang, Yong-Jun Tan, Rui Luo, Ye Qiu, Xing-Yi Ge, The ORF6, ORF8 and nucleocapsid proteins of SARS-COV-2 inhibit type I interferon signaling pathway, Virus Research, Volume 286, 2020,
Khailany-Khailany R A, Safdar M, Ozaslan M. Genomic characterization of a novel SARS-COV-2 [published online ahead of print, 2020 Apr. 16]. Gene Rep. 2020; 19:100682. doi: 10.1016/j.genrep.2020.100682
T. Koyama, D. Platt, L. Parida, Variant analysis of COVID-19 genomes, Bull. World Health Organ. (2020)
Catanzaro, M., Fagiani, F., Racchi, M., Corsini, E., Govoni, S., Lanni, C., 2020. Immune response in COVID-19: addressing a pharmacological challenge by targeting pathways triggered by SARS-COV-2. Signal Transduct. Target. Ther. 5 (1), 84.
Seema Mishra-Mishra, Seema (2020): ORF10: Molecular Insights into the Contagious Nature of Pandemic Novel Coronavirus 2019-nCOV. ChemRxiv. Preprint. https://doi.org/10.26434/chemrxiv.12118839.v3
Xu, J. et al., Viruses 2020, 12, 244
Tang, X. et al. National Science Review 2020, 7, 1012-1023
M. Bianchi et al, BioMed Research International Vol 2020 Article ID 4389089
Hassan S, Eldeeb K, Millns P J, Bennett A J, Alexander S P, Kendall D A. Cannabidiol enhances microglial phagocytosis via transient receptor potential (TRP) channel activation. Br J Pharmacol. 2014; 171(9):2426-39.
da Silva V K, de Freitas B S, da Silva Dornelles A, Nery L R, Falavigna L, Ferreira R D, et al. Cannabidiol normalizes caspase 3, synaptophysin, and mitochondrial fission protein DNMIL expression levels in rats with brain iron overload: implications for neuroprotection. Molecular neurobiology. 2014; 49(1):222-33.
Huang Y, Wan T, Pang N, Zhou Y, Jiang X, Li B, et al. Cannabidiol protects livers against nonalcoholic steatohepatitis induced by high-fat high cholesterol diet via regulating NF-κB and NLRP3 inflammasome pathway. J Cell Physiol. 2019; 234(11):21224-34.
Hassan S, Eldeeb K, Millns P J, Bennett A J, Alexander S P, Kendall D A. Cannabidiol enhances microglial phagocytosis via transient receptor potential (TRP) channel activation. Br J Pharmacol. 2014; 171(9):2426-39.
da Silva V K, de Freitas B S, Dornelles V C, Kist L W, Bogo M R, Silva M C, et al. Novel insights into mitochondrial molecular targets of iron-induced neurodegeneration: Reversal by cannabidiol. Brain Res Bull. 2018; 139:1-8.
Hao E, Mukhopadhyay P, Cao Z, Erdelyi K, Holovac E, Liaudet L, et al. Cannabidiol Protects against Doxorubicin-Induced Cardiomyopathy by Modulating Mitochondrial Function and Biogenesis. Mol Med. 2015; 21:38-45.
McKallip R J, Jia W, Schlomer J, Warren J W, Nagarkatti P S, Nagarkatti M. Cannabidiol-induced apoptosis in human leukemia cells: A novel role of cannabidiol in the regulation of p22phox and Nox4 expression. Mol Pharmacol. 2006; 70(3):897-908.
Ryan D, Drysdale A J, Lafourcade C, Pertwee R G, Platt B. Cannabidiol targets mitochondria to regulate intracellular Ca2+ levels. J Neurosci. 2009; 29(7):2053-63.
Valvassori S S, Bavaresco D V, Scaini G, Varela R B, Streck E L, Chagas M H, et al. Acute and chronic administration of cannabidiol increases mitochondrial complex and creatine kinase activity in the rat brain. Braz J Psychiatry. 2013; 35(4):380-6.
Jeong S, Jo M J, Yun H K, Kim D Y, Kim B R, Kim J L, et al. Cannabidiol promotes apoptosis via regulation of XIAP/Smac in gastric cancer. Cell Death Dis. 2019; 10(11):846.
Jeong S, Yun H K, Jeong Y A, Jo M J, Kang S H, Kim J L, et al. Cannabidiol-induced apoptosis is mediated by activation of Noxa in human colorectal cancer cells. Cancer letters. 2019; 447:12-23.
Sultan A S, Marie M A, Sheweita S A. Novel mechanism of cannabidiol-induced apoptosis in breast cancer cell lines. Breast. 2018; 41:34-41.
Oláh A, Markovics A, Szabó-Papp J, Szabó P T, Stott C, Zouboulis C C, et al. Differential effectiveness of selected non-psychotropic phytocannabinoids on human sebocyte functions implicates their introduction in dry/seborrhoeic skin and acne treatment. Exp Dermatol. 2016; 25(9):701-7.
Solinas M, Massi P, Cantelmo A R, Cattaneo M G, Cammarota R, Bartolini D, et al. Cannabidiol inhibits angiogenesis by multiple mechanisms. Br J Pharmacol. 2012; 167(6): 1218-31.
Castillo A, Tolon M R, Fernandez-Ruiz J, Romero J, Martinez-Orgado J. The neuroprotective effect of cannabidiol in an in vitro model of newborn hypoxic-ischemic brain damage in mice is mediated by CB(2) and adenosine receptors. Neurobiology of disease. 2010; 37(2):434-40.
Sun S, Hu F, Wu J, Zhang S. Cannabidiol attenuates OGD/R-induced damage by enhancing mitochondrial bioenergetics and modulating glucose metabolism via pentose-phosphate pathway in hippocampal neurons. Redox Biol. 2017; 11:577-85.
Burstein S, Hunter S A, Renzulli L. Stimulation of sphingomyelin hydrolysis by cannabidiol in fibroblasts from a Niemann-Pick patient. Biochemical and biophysical research communications. 1984; 121(1): 168-73.
Cornicelli J A, Gilman S R, Krom B A, Kottke B A. Cannabinoids impair the formation of cholesteryl ester in cultured human cells. Arteriosclerosis. 1981; 1(6):449-54.
Rimmerman N, Bradshaw H B, Kozela E, Levy R, Juknat A, Vogel Z. Compartmentalization of endocannabinoids into lipid rafts in a microglial cell line devoid of caveolin-1. Br J Pharmacol. 2012; 165(8): 2436-49.
Lu Y et al 2008-45. Lu Y, Liu D X, Tam J P. Lipid rafts are involved in SARS-CoV entry into Vero E6 cells. Biochemical and biophysical research communications. 2008; 369(2):344-9.
Duncan R E, El-Sohemy A, Archer M C. Mevalonate promotes the growth of tumors derived from human cancer cells in vivo and stimulates proliferation in vitro with enhanced cyclin-dependent kinase-2 activity. The Journal of biological chemistry. 2004; 279(32): 33079-84.
Duncan R E, Lau D, El-Sohemy A, Archer M C. Geraniol and beta-ionone inhibit proliferation, cell cycle progression, and cyclin-dependent kinase 2 activity in MCF-7 breast cancer cells independent of effects on HMG-COA reductase activity. Biochemical pharmacology. 2004; 68(9): 1739-47.
Gordon, D. E., Jang, G. M., Bouhaddou, M. et al. A SARS-COV-2 protein interaction map reveals targets for drug repurposing. Nature 583, 459-468 (2020). https://doi.org/10.1038/s41586-020-2286-9
Bizzotto J, Sanchis P, Abbate M, Lage-Vickers S, Lavignolle R, Toro A, Olszevicki S, Sabater A, Cascardo F, Vazquez E, Cotignola J, Gueron G. SARS-COV-2 Infection Boosts MX1 Antiviral Effector in COVID-19 Patients. iScience. 2020 Oct. 23; 23(10):101585.
Zhou S, Butler-Laporte G, Nakanishi T, Morrison D R, Afilalo J, Afilalo M, Laurent L, Pietzner M, Kerrison N, Zhao K, Brunet-Ratnasingham E, Henry D, Kimchi N,
Afrasiabi Z, Rezk N, Bouab M, Petitjean L, Guzman C, Xue X, Tselios C, Vulesevic B, Adeleye O, Abdullah T, Almamlouk N, Chen Y, Chassé M, Durand M, Paterson C, Normark J, Frithiof R, Lipcsey M, Hultström M, Greenwood CMT, Zeberg H, Langenberg C, Thysell E, Pollak M, Mooser V, Forgetta V, Kaufmann D E, Richards J B. A Neanderthal OAS1 isoform protects individuals of European ancestry against COVID-19 susceptibility and severity. Nat Med. 2021 Feb. 25.
Duncan, R. E., et al., Geraniol and beta-ionone inhibit proliferation, cell cycle progression, and cyclin-dependent kinase 2 activity in MCF-7 breast cancer cells independent of effects on HMG-COA reductase activity. Biochem Pharmacol, 2004. 68(9): p. 1739-47.
M'Hiri I, Diaguarachchige De Silva K H, Duncan R E. Relative expression and regulation by short-term fasting of lysophosphatidic acid receptors and autotaxin in white and brown adipose tissue depots. Lipids. 2020; 55(3):279-84.
Nagarkatti P, Pandey R, Rieder S A, Hegde V L, Nagarkatti M. Cannabinoids as novel anti-inflammatory drugs. Future Med Chem. 2009; 1(7): 1333-1349. doi: 10.4155/fmc.09.93.

1. A pharmaceutical composition comprising therapeutically effective amount of Cannabidiol for use in treatment of Covid-19 infectious disease caused by Sars-Cov-2 virus wherein administration of said pharmaceutical composition to the said patient suffering from Covid-19 produces an enhancement/augmentation of innate immunity of the patient due to at least one of the following effects,
i) infected patient cells undergo apoptosis early after infection;
ii) induction of interferon transcription in the patient;
iii) induction of interferon-induced antiviral effectors in the patient.

2. The pharmaceutical composition according to claim 1 wherein the enhancement/augmentation of innate immunity of the patient is due to apoptosis of the infected patient cells early after infection which renders them not available
to the virus for replication and/or mutation.

3. The pharmaceutical composition according to claim 2 wherein the enhancement/augmentation of innate immunity of the patient is due to a late apoptosis of the infected cells of the patient in addition to the apoptosis early after infection.

4. The pharmaceutical composition according to claim 2 wherein the
enhancement/augmentation of innate immunity of the patient is due to the apoptosis of the infected cells of the patient early after infection or apoptosis of the infected cells of the patient early after infection as well as late apoptosis of the infected cells of the patient thereby reducing or abolishing ability of the virus to evade host immunity.

5. The pharmaceutical composition according to claim 1 wherein the enhancement/augmentation of innate immunity of the patient is due to induction of interferon transcription in the patient providing an innate, intracellular, anti-viral defense.

6. The pharmaceutical composition according to claim 5 wherein the enhancement/augmentation of innate immunity of the patient is due to induction of Type II (gamma) or Type III (lambda) or both Type II and Type III Interferon transcription in such patients.

7. The pharmaceutical composition according to claim 1 wherein the enhancement/augmentation of innate immunity of the patient is due to induction of interferon induced antiviral effector in the patient wherein the antiviral effector is one or more of OAS1, Mx1 and IFIT1 genes.

8. The pharmaceutical composition according to claim 1 wherein the enhancement/augmentation of innate immunity of the patient is due to induction of interferon induced antiviral effector in the patient wherein the antiviral effector is OAS1 gene.

9. A pharmaceutical composition comprising therapeutically effective amount of Cannabidiol for use in prophylaxis or prophylactic treatment of Covid-19 wherein administration of said pharmaceutical composition to a mammal/human produces an enhancement/augmentation of innate immunity in such mammal/human due to at least one of the following effects,
i) induction of interferon transcription in the mammal/human;
ii) induction of interferon-induced antiviral effectors in the mammal/human.

10. The pharmaceutical composition of claim 9 wherein enhancement/augmentation of innate immunity in mammal/human is not associated with apoptosis of cells.

11. The pharmaceutical composition of claim 10 wherein enhancement/augmentation of innate immunity in mammal/human is due to induction of Type II (gamma) or Type III (lambda) or both Type II and Type III Interferon transcription.

12. The pharmaceutical composition of claim 10 wherein enhancement/augmentation of innate immunity in mammal/human is due to induction of interferon induced antiviral effector wherein the antiviral effector is one or more of OAS1, Mx1 and IFIT1 genes.

13. The pharmaceutical composition of claim 10 wherein enhancement/augmentation of innate immunity in mammal/human is due to induction of interferon induced antiviral effector wherein the antiviral effector is OAS1 gene.

14. A method of treating Covid-19 infectious disease caused by Sars-Cov-2 virus wherein said method comprises administering a pharmaceutical composition comprising therapeutically effective amount of cannabidiol to a patient wherein the administration of said pharmaceutical composition to the said patient produces an
enhancement/augmentation of innate immunity of the patient due to at least one of the following effects,
i) infected patient cells undergo apoptosis early after infection;
ii) induction of interferon transcription in the patient;
iii) induction of interferon induced antiviral effector in the patient.

15. The method of treating Covid-19 according to claim 14 wherein the enhancement/augmentation of innate immunity of the patient is due to the apoptosis of infected patient cells early after infection which renders them not available to the virus for replication and/or mutation.

16. The method of treating Covid-19 according to claim 12 wherein the enhancement/augmentation of innate immunity of the patient is due to the late apoptosis of the infected cells of the patient in addition to the apoptosis of infected patient cells early after infection.

17. The method of treating Covid-19 according to claim 12 wherein the enhancement/augmentation of innate immunity of the patient is due to the apoptosis of infected patient cells early after infection or apoptosis of infected patient cells early after infection as well as late apoptosis of the infected cells of the patient, thereby reducing or abolishing ability of the virus to evade host immunity.

18. The method of treating Covid-19 according to claim 14 wherein the enhancement/augmentation of innate immunity of the patient is due to induction of interferon transcription in the patient providing an innate, intracellular, anti-viral defense.

19. The method of treating Covid-19 according to claim 14 wherein the enhancement/augmentation of innate immunity of the patient is due to induction of Type II (gamma) or Type III (lambda) or both Type II and Type III Interferon transcription in such patients.

20. The method of treating Covid-19 according to claim 14 wherein the enhancement/augmentation of innate immunity of the patient is due to induction of interferon-induced antiviral effector in the patient wherein the antiviral effector is one or more of OAS1, Mx1 and IFIT1 genes.

21. The method of treating Covid-19 according to claim 14 wherein the enhancement/augmentation of innate immunity of the patient is due to induction of interferon-induced antiviral effector in the patient wherein the antiviral effector is OAS1 gene.

22. A method of prophylaxis or prophylactic treatment of Covid-19 infectious disease caused by Sars-Cov-2 virus wherein said method comprises administering a pharmaceutical composition comprising therapeutically effective amount of Cannabidiol to a mammal/human wherein administration of said pharmaceutical composition produces an enhancement/augmentation of innate immunity in such mammal/human due to at least one of the following effects,
i) induction of interferon transcription in the mammal/human;
ii) induction of interferon-induced antiviral effectors in the mammal/human.

23. The method of prophylaxis or prophylactic treatment of claim 22 wherein enhancement/augmentation of innate immunity in mammal/human is not associated with apoptosis of cells.

24. The method of prophylaxis or prophylactic treatment of claim 23 wherein enhancement/augmentation of innate immunity in mammal/human is due to induction of Type II (gamma) or Type III (lambda) or both Type II and Type III Interferon transcription.

25. The method of prophylaxis or prophylactic treatment of claim 23 wherein enhancement/augmentation of innate immunity in mammal/human is due to induction of interferon induced antiviral effector wherein the antiviral effector is one or more of OAS1, Mx1 and IFIT1 genes.

26. The method of prophylaxis or prophylactic treatment of claim 23 wherein enhancement/augmentation of innate immunity in mammal/human is due to induction of interferon induced antiviral effector wherein the antiviral effector is OAS1 gene.

27. A pharmaceutical composition comprising therapeutically effective amount of Cannabidiol for preventing or reducing mutation of Sars-Cov-2 virus in a patient by administration of said pharmaceutical composition to the said patient suffering from Covid-19 by causing infected patient cells to undergo apoptosis early after infection which renders them not available to the virus for mutation.

28. A method of preventing or reducing mutation of Sars-Cov-2 virus in a patient wherein said method comprises administering a pharmaceutical composition comprising therapeutically effective amount of cannabidiol to a patient suffering from Covid-19 by causing infected patient cells to undergo apoptosis early after infection which renders them not available to the virus for mutation.

29. A pharmaceutical composition comprising therapeutically effective amount of Cannabidiol for use in preventing or better preparing for Covid-19 infectious disease in mammals/humans who are about to get infected with Covid-19 infectious disease wherein administration of said pharmaceutical composition to the
mammal/human produces an enhancement/augmentation of innate immunity in such mammal/human due to at least one of the following effects,
i) induction of interferon transcription in the mammal/human;
ii) induction of interferon-induced antiviral effectors in the mammal/human wherein such induction is not associated with apoptosis of cells when virus is not present but enables cells to get primed/prepared for viral threat and wherein such cells are better able to prepare for infection including by causing an increase in the infectious dose of virions to the organism than the usual dose required to cause disease and wherein the cells undergo apoptosis early after infection which renders the cells not available to the virus for replication and/or mutation.

30. A method of preventing or better preparing for Covid-19 infectious disease in mammals/humans who are about to get infected with Covid-19 infectious disease wherein said method comprises administering a pharmaceutical composition comprising therapeutically effective amount of cannabidiol to the mammal/human wherein administration of said pharmaceutical composition to the mammal/human produces an enhancement/augmentation of innate immunity in such mammal/human due to at least one of the following effects,
i) induction of interferon transcription in the mammal/human;
ii) induction of interferon-induced antiviral effectors in the mammal/human;
wherein such induction is not associated with apoptosis of cells when virus is not present but enables cells to get primed/prepared for viral threat and wherein such cells are better able to prepare for infection including by causing an increase in the infectious dose of virions to the organism than usual dose required to cause disease and wherein the cells undergo apoptosis early after infection which renders the cells not available to the virus for replication and/or mutation.


SEQUENCE LISTING (15 sequences)

SEQ ID NO: 1 - DNA, 20 bp, Homo sapiens
tggcttttcagctctgcatc

SEQ ID NO: 2 - DNA, 21 bp, Homo sapiens
ccgctacatctgaatgacctg

SEQ ID NO: 3 - DNA, 20 bp, Homo sapiens
gaggcccccaaaaaggagtc

SEQ ID NO: 4 - DNA, 20 bp, Homo sapiens
aggttcccatcggccacata

SEQ ID NO: 5 - DNA, 20 bp, Homo sapiens
ctgccacatagcccagttca

SEQ ID NO: 6 - DNA, 25 bp, Homo sapiens
agaagcgactcttctaaggcatctt

SEQ ID NO: 7 - DNA, 22 bp, Homo sapiens
ggctgtttaccagactccgaca

SEQ ID NO: 8 - DNA, 22 bp, Homo sapiens
cacaaagcctggcagctctcta

SEQ ID NO: 9 - DNA, 19 bp, Homo sapiens
gaaggcagctcacgaaacc

SEQ ID NO: 10 - DNA, 18 bp, Homo sapiens
aggcctcagcctcttgtg

SEQ ID NO: 11 - DNA, 21 bp, Homo sapiens
ggaatacacaacctactagcc

SEQ ID NO: 12 - DNA, 20 bp, Homo sapiens
ccaggtcaccagactcctca

SEQ ID NO: 13 - DNA, 20 bp, Homo sapiens
agaaggctggggctcatttg

SEQ ID NO: 14 - DNA, 20 bp, Homo sapiens
aggggccatccacagtcttc

SEQ ID NO: 15 - protein, 24 aa, Homo sapiens
DYKDDDDKDYKDDDDKDYKDDDDK